(19) United States
(12) Patent Application Publication
(10) Pub. No.: US 2026/0126444 A1
(43) Pub. Date: May 7, 2026

(54) Biomarkers for Long COVID

(71) Applicant: Szegedi Tudományegyetem, Szeged (HU)

(72) Inventors: Tibor PANKOTAI, Domaszék (HU); László TISZLAVICZ, Makó (HU); Barbara Nikolett BORSOS, Szeged (HU)

(21) Appl. No.: 19/383,282

(22) Filed: Nov. 7, 2025

(30) Foreign Application Priority Data
Nov. 7, 2024 (HU) P2400508
Nov. 8, 2024 (HU) P2400512

(51) Int. Cl. G01N 33/573; A61K 31/09; A61K 31/155; A61K 31/436; G01N 33/68

(52) U.S. Cl. CPC G01N 33/573; A61K 31/09; A61K 31/155; A61K 31/436; G01N 33/6893; C12Y 115/01001; C12Y 306/05005; G01N 2333/90283; G01N 2333/96466; G01N 2800/52

(57) ABSTRACT

The present invention relates to a method for in vitro diagnosis of Long COVID in a subject, wherein the method comprises the following steps: a) providing a biological sample obtained from the subject; b) measuring the levels of at least one protein in said sample, wherein the at least one protein is selected from Autophagy Related 4B Cysteine Peptidase (ATG4B), Mitofusin 2 (MFN2), Dynamin-related Protein 1 (DRP1), and/or Superoxide dismutase 1 (SOD1); and c) comparing the levels of the at least one protein measured in step b) with a respective reference, wherein an increase in the levels of the at least one protein in said sample relative to the reference is indicative of Long COVID diagnosis.

This application claims priority to Hungarian Application No. P2400508 filed Nov. 7, 2024, and Hungarian Application No. P2400512 filed Nov. 8, 2024, which are both herein incorporated in their entirety by reference.

The content of the sequence listing named 124512-00067_sequence_listing_editl.xml, which is 11,150 bytes in size was created on Nov. 7, 2025, and is incorporated herein by reference in its entirety.

### FIELD OF THE INVENTION

The present invention relates to methods for in vitro diagnosis of Long COVID in a subject, using quantified or detected biomarkers in a biological sample. In particular, the invention relates to a method for in vitro diagnosis of Long COVID in a subject, wherein said method comprises the following steps: a) providing a biological sample obtained from the subject; b) measuring the levels of at least one protein in said sample, wherein the at least one protein is selected from Autophagy Related 4B Cysteine Peptidase (ATG4B), Mitofusin 2 (MFN2), Dynamin-related Protein 1 (DRP1), and/or Superoxide dismutase 1 (SOD1); and c) comparing the levels of the at least one protein measured in step b) with a respective reference, wherein an increase in the levels of the at least one protein in said sample relative to the reference is indicative of Long COVID diagnosis.

### TECHNICAL BACKGROUND

The emergence of Coronavirus Disease 2019 (COVID-19), caused by the severe acute respiratory syndrome coronavirus 2 (SARS-CoV-2), has precipitated a global health crisis with enduring implications. As of the latest updates, COVID-19 has affected over 775 million individuals worldwide, resulting in more than 7 million deaths across various countries and territories [1]. The mortality rate for COVID-19 differs significantly by age, with older adults, especially those with underlying health conditions, experiencing disproportionately higher rates of fatalities [2-5]. The pandemic has seen multiple waves, driven by the emergence of virus variants, each varying in transmissibility and virulence [6-7]. Despite extensive vaccination efforts, which have seen billions of vaccine doses administered globally, the virus continues to impact populations, healthcare systems, and economies.

While the majority of affected individuals recover from the acute respiratory syndrome within a few weeks, approximately 30-70% of those infected experience persistent and debilitating symptoms collectively termed Long COVID, post-COVID-19 syndrome, or Post-Acute Sequelae of SARS-CoV-2 infection (PASC) [3, 8-26]. Chronic fatigue is consistently identified as the most common and debilitating symptom reported by survivors, as demonstrated by various cross-sectional and cohort studies [18, 27-31]. Individuals affected by Long COVID often experience a broad range of additional symptoms, including dyspnea, joint pain, sleep problems, mood disorders such as depression and anxiety [32], headaches, dizziness, cognitive issues commonly referred to as “brain fog”, and cardiac symptoms [18]. These symptoms can persist for months and significantly impair quality of life. The National Institute for Health and Care Excellence categorizes PASC as ongoing symptomatic COVID-19 for individuals whose symptoms persist between four and twelve weeks following the initial onset of acute symptoms or as post-COVID-19 syndrome for those whose symptoms continue beyond twelve weeks [18, 33]. In contrast, the World Health Organization describes PASC as a condition affecting individuals with a suspected or confirmed SARS-CoV-2 infection who experience lasting symptoms for a minimum of two months, and where these symptoms cannot be attributed to another underlying medical condition [9, 34].

Long COVID presents a complex clinical picture that implicates multiple organ systems. Emerging evidence suggests mitochondrial dysfunction as a central component of this syndrome [35-49]. Mitochondria, essential for energy production and cellular metabolism, are particularly vulnerable to SARS-CoV-2 infection [36]. The virus may hijack and reprogram mitochondrial function or inflict direct damage through various mechanisms during and potentially after infection [36]. Such disruptions lead to altered energy metabolism, which is believed to contribute to the fatigue, cognitive impairments, and muscular weaknesses commonly observed in Long COVID patients [35, 36].

Siekacz et al. [83]investigated the association between post-COVID-19 pulmonary complications and mitochondrial regulatory proteins in the context of oxidative stress. They found that serum concentrations of mitochondrial regulatory proteins (e.g. PINKI, DNM1L, MFN2) were significantly higher in the long-term pulmonary complications group. They concluded that SARS-CoV-2 infection could be involved in mitochondrial imbalance via PINKI, DNM1L, and MFN2 dysregulation, however, they suggested only that TNF-α might be a potential predictor of pulmonary complications.

US20240219385A1 discloses a method of diagnosing or classifying a subject as having a chronic or long-term infection of a virus (e.g. SARS-CoV-2), which comprises determining the level of one or more biomarkers in a biological sample obtained from a subject. The biomarkers can be proteins associated with an inflammatory response (e.g. TNF, IFNLR1, BCAM, S100A16, IL5), or hormones and hormone receptors (e.g. CRH, CRHR1, PTH1R), or transcription factors and motifs thereof (e.g. AP-1, BACH, BATF, IRF, STAT).

WO2023147669A1 discloses a method for diagnosing Long-COVID in a patient, in which the measured levels of one or more biomarkers selected from ANG-1, P-Sel and MMP-1 is/are used for the diagnosis.

WO2024065059A1 discloses a method of diagnosing long-COVID-19 in a patient, in which the detected levels of one or more proteins are used for the diagnosis. They listed 119 protein (e.g. CXCL5, AP3S2, MAX, PDLIM7, ED AR, LTA4H, CRACR2A, CXCL3, FRZB) that can be used for diagnosis.

WO2024124357A1 discloses methods of diagnosing Long-COVID patients, and methods for determining the risk of developing certain disorders/injuries in Long-COVID patients. One method is for diagnosing Long-COVID in a patient, in which the increased levels of at least one marker (selected from C3, PLCB1, HLA-DRA, CD 177, CCL5, PLCB2, CR1, NFKB, NCAM1/CD56, IFNG, IL-22, SELP, CASP1, PADI4, ITGB2, TLR2, CD14, CCL8, PDGFA, PDGFRAB, HIFa, IGFBP6, IGFBPL1, APP, JAM2, KCNH2, S100A14, KIAA0319, SNAPIN, and ITGAM) compared to healthy controls indicates Long-COVID. In another method, the level of resting natural killer cells in a sample is used for diagnosing a patient with Long-COVID.

Despite that biomarkers are suggested for the diagnosis of Long COVID in the art, there is still a need for reliable biomarkers that can be used in the diagnosis of this complicated syndrome with very different and various symptoms.

### BRIEF DESCRIPTION OF THE INVENTION

The present invention relates to a method for in vitro diagnosis of Long COVID in a subject, wherein said method comprises the following steps:
a) providing a biological sample obtained from the subject,
b) measuring the levels of at least one protein in said sample,
   wherein the at least one protein is selected from Autophagy Related 4B Cysteine Peptidase (ATG4B), Mitofusin 2 (MFN2), Dynamin-related Protein 1 (DRP1), and/or Superoxide dismutase 1 (SOD1),
c) comparing the levels of the at least one protein measured in step b) with a respective reference, wherein an increase in the levels of the at least one protein in said sample relative to the reference is indicative of Long COVID diagnosis.

Preferably, the at least one protein is at least two proteins selected from ATG4B, MFN2, DRP1, and/or SOD1.

Preferably, the at least one protein is at least three proteins selected from ATG4B, MFN2, DRP1, and SOD1.

Preferably, the at least one protein is the following four proteins: ATG4B, MFN2, DRP1, and SOD1.

Preferably, wherein the levels of at least two proteins are measured, an increase in the levels of at least one of the proteins in said sample relative to the respective reference is indicative of Long COVID diagnosis. Preferably, the levels of at least two proteins are measured in step b), and an increase in the levels of at least two of the proteins is indicative of Long COVID diagnosis. Preferably, the at least two proteins are selected from ATG4B, MFN2, DRP1, and/or SOD1.

Preferably, in any of the methods, the biological sample of step a) is a tissue sample or a sample derived from circulating biological fluid. More preferably, the biological sample is a tissue sample. More preferably, the tissue sample is a biopsy sample. More preferably, the biopsy sample is a nasal mucosal or bronchial biopsy sample.

Preferably, the subject is a vertebrate subject, preferably a mammalian subject, more preferably a human subject.

Preferably, the reference is the levels of the at least one protein measured in sample(s) obtained from a healthy subject or a group of healthy subjects, i.e. a subject or a group of subjects not suffering from long COVID.

In another embodiment, the method for in vitro diagnosis of Long COVID in a subject comprises the following steps:
a) providing a biological sample obtained from the subject,
b) measuring the levels of at least one protein in the sample of step a),
   wherein the at least one protein is selected from ATG4B, MFN2, DRP1, and/or SOD1,
c) comparing the levels of the at least one protein measured in step b) with a respective reference,
d) providing another biological sample obtained from the subject,
e) measuring the levels of circulating cell-free mitochondrial DNA (ccf-mtDNA) in the sample of step d),
f) comparing the levels of ccf-mtDNA measured in step e) with a respective reference,
wherein an increase in the levels of the at least one protein in the sample of step a) relative to the respective reference and/or a decrease in the levels of ccf-mtDNA in the sample of step d) relative to the respective reference is indicative of Long COVID diagnosis.

Preferably, the at least one protein is at least two proteins selected from ATG4B, MFN2, DRP1, and SOD1.

Preferably, the at least one protein is at least three proteins selected from ATG4B, MFN2, DRP1, and SOD1.

Preferably, the at least one protein is the following four proteins: ATG4B, MFN2, DRP1, and SOD1.

Preferably, the circulating cell-free mitochondrial DNA measured in step e) may be any DNA segment of mitochondrial DNA, preferably wherein the DNA segment is at least 100 bp long, more preferably at least 200 bp long.

Preferably, the circulating cell-free mitochondrial DNA measured in step e) is selected from the group comprising MTATP6-, MTCYTB-, MTND1-, MTND4-, and MTND5-specific DNAs. Preferably, the circulating cell-free mitochondrial DNA measured in step e) is selected from the group consisting of MTATP6-, MTCYTB-, MTND1-, MTND4-, and MTND5-specific DNAs. Preferably, the ccf-mtDNA measured in step e) is at least one of the following: MTATP6-, MTCYTB-, MTND1-, MTND4-, and/or MTND5-specific DNAs.

Preferably, the ccf-mtDNA comprises at least two different mitochondrial DNA segments or mitochondrial genes, more preferably at least three different mitochondrial DNA segments or mitochondrial genes, more preferably at least four different mitochondrial DNA segments or mitochondrial genes, even more preferably at least five different mitochondrial DNA segments or mitochondrial genes. More preferably, the mitochondrial DNA segments or genes are selected from MTATP6-, MTCYTB-, MTND1-, MTND4-, and MTND5-specific DNAs.

Preferably, the ccf-mtDNA is at least two different mitochondrial DNA segments or mitochondrial genes, wherein the mitochondrial DNA is selected from MTATP6-, MTCYTB-, MTND1-, MTND4-, and MTND5-specific DNAs.

Preferably, the ccf-mtDNA is at least three different mitochondrial DNA segments or mitochondrial genes, wherein the mitochondrial DNA is selected from MTATP6-, MTCYTB-, MTND1-, MTND4-, and MTND5-specific DNAs.

Preferably, the ccf-mtDNA is at least four different mitochondrial DNA segments or mitochondrial genes, wherein the mitochondrial DNA is selected from MTATP6-, MTCYTB-, MTND1-, MTND4-, and MTND5-specific DNAs.

Preferably, the ccf-mtDNA is five different mitochondrial DNA segments or mitochondrial genes, wherein the five mitochondrial DNAs are MTATP6-, MTCYTB-, MTND1-, MTND4-, and MTND5-specific DNAs.

Preferably, wherein the ccf-mtDNA comprises at least two mitochondrial DNA segments or mitochondrial genes, a decrease in the levels of at least one of them is indicative of Long COVID diagnosis.

Preferably, the presence of at least one of the following is indicative of Long COVID diagnosis: an increase in the levels of at least one of the proteins compared to the respective reference and/or a decrease in the levels of at least one of the measured ccf-mtDNA compared to the respective reference. Preferably, an increase in the levels of at least two proteins compared to the respective reference and/or a decrease in the levels of at least one of the ccf-mtDNA compared to the respective reference is indicative of Long COVID diagnosis.

Preferably, the biological sample of step a) is a tissue sample, preferably a biopsy, more preferably a nasal mucosal or bronchial biopsy sample.

Preferably, the biological sample of step d) is a sample derived from circulating biological fluid. More preferably, the biological sample is selected from blood, serum, plasma or cerebrospinal fluid. More preferably, the biological sample is blood, serum, or plasma.

Preferably, the subject is a vertebrate subject, preferably a mammalian subject, more preferably a human subject.

Preferably, the reference is the levels of protein(s) or ccf-mtDNA, respectively, measured in sample(s) obtained from a healthy subject or a group of healthy subjects, i.e. a subject or a group of subjects not suffering from long COVID.

Preferably, the subject is a vertebrate subject, preferably a mammalian subject, more preferably a human subject.

In another embodiment, the method for in vitro diagnosis of Long COVID in a subject comprises the following steps:
a) providing a biological sample obtained from the subject,
b) measuring the levels of at least one protein in the sample of step a),
   wherein the at least one protein is selected from ATG4B, MFN2, DRP1, and/or SOD1,
c) comparing the levels of the at least one protein measured in step b) with a respective reference,
d) providing another biological sample obtained from the subject,
e) measuring the levels of circulating cell-free mitochondrial DNA (ccf-mtDNA) in the sample of step d),
f) comparing the levels of ccf-mtDNA measured in step e) with a respective reference,
g) optionally providing another biological sample obtained from the subject,
h) detecting one or more mitochondrial features in the sample of step a) or step g), wherein the mitochondrial feature is selected from mitochondrial structural features (mitochondrial morphology), number of mitochondria, and/or size of mitochondria,
i) comparing the one or more mitochondrial features detected in step h) with a respective reference,
wherein an increase in the levels of the at least one protein in the sample of step a) relative to the respective reference and/or a decrease in the levels of ccf-mtDNA in the sample of step d) relative to the reference and/or a difference in the detected one or more mitochondrial features in the sample of step g) or step a) relative to the respective reference is indicative of Long COVID diagnosis.

Preferably, the at least one protein is as defined above.

Preferably, the ccf-mtDNA is as defined above.

Preferably, the biological sample of step a) and/or step g) is a tissue sample, preferably a biopsy, more preferably a nasal mucosal or bronchial biopsy sample.

Preferably, the biological sample of step d) is a sample derived from circulating biological fluid. More preferably, the biological sample is selected from blood, serum, plasma or cerebrospinal fluid. More preferably, the biological sample is blood, serum, or plasma.

Preferably, the subject is a vertebrate subject, preferably a mammalian subject, more preferably a human subject.

Preferably, the reference is the levels of protein(s) or ccf-mtDNA or mitochondrial feature(s), respectively, measured or detected in sample(s) of a healthy subject or a group of healthy subjects, i.e. a subject or a group of subjects not suffering from long COVID.

The present invention further relates to a method for in vitro diagnosis of Long COVID in a subject, wherein said method comprises the following steps:
a) providing a biological sample obtained from the subject,
b) quantifying or detecting one or more biomarkers in said sample,
c) comparing the quantified or detected one or more biomarkers in said sample with a reference, wherein a difference in the quantified or detected one or more biomarkers in said sample relative to the respective reference is indicative of Long COVID diagnosis,
wherein the one or more biomarkers are selected from:
   mitochondrial structural features (mitochondrial morphology), number of mitochondria, and/or size of mitochondria; and/or
   proteins selected from Autophagy Related 4B Cysteine Peptidase (ATG4B), Mitofusin 2 (MFN2), Dynamin-related Protein 1 (DRP1), and/or Superoxide dismutase 1 (SOD1); and/or
   circulating cell-free mitochondrial DNA (ccf-mtDNA).

Preferably, the ccf-mtDNA is as defined above.

Preferably, the protein is at least one protein as defined above.

Preferably, the subject is a vertebrate subject, preferably a mammalian subject, more preferably a human subject.

Preferably, the biological sample is a tissue sample or a sample derived from circulating biological fluid. More preferably, the biological sample is selected from biopsy, blood, serum, plasma or cerebrospinal fluid. More preferably, the biological sample is biopsy, blood, serum, or plasma.

Preferably, the reference is the levels of protein(s) or ccf-mtDNA or mitochondrial feature(s), respectively, measured in sample(s) obtained from a healthy subject or a group of healthy subjects, i.e. a subject or a group of subjects not suffering from long COVID.

The present invention further relates to a method of monitoring the treatment of Long COVID in a subject, wherein said method comprises the following steps:
a) providing at least one biological sample obtained from the subject undergoing Long COVID treatment,
   wherein the sample(s) are obtained after the long COVID treatment has begun,
b) measuring the levels of at least one protein in said sample(s),
   wherein the at least one protein is selected from ATG4B, MFN2, DRP1, and/or SOD1,
c) comparing the levels of the at least one protein measured in step b) with a reference, wherein a similarity in the levels of the at least one protein in said sample relative to the reference indicates an effective Long COVID treatment.

In another embodiment, the method of monitoring the treatment of Long COVID in a subject comprises the following steps:
a) providing at least two biological samples obtained from the subject undergoing Long COVID treatment,
   wherein one sample is obtained prior to the start of treatment and the other sample(s) are obtained after the treatment has begun,
b) measuring the levels of at least one protein in the sample obtained prior to the start of the treatment,
   wherein the at least one protein is selected from ATG4B, MFN2, DRP1, and/or SOD1,
   thereby obtaining a baseline,
c) measuring the levels of the same protein(s) as in step b) in the sample(s) obtained after the treatment has begun,
d) comparing the levels of the protein(s) measured in step c) with the baseline obtained in step b), wherein an increase in the levels of the protein(s) in the sample(s) obtained after the treatment has begun, relative to the baseline, indicates an effective Long COVID treatment.

Preferably, the monitoring is carried out in vitro.

Preferably, the at least one protein is as defined above.

Preferably, the biological sample is a tissue sample, preferably a biopsy, more preferably a nasal mucosal or bronchial biopsy sample.

Preferably, the reference is the levels of the at least one protein measured in sample(s) obtained from a healthy subject or a group of healthy subjects, i.e. a subject or a group of subjects not suffering from long COVID.

Preferably, the subject is a vertebrate subject, preferably a mammalian subject, more preferably a human subject.

The present invention also relates to a protein for use in the treatment of Long COVID in a subject, wherein said protein is selected from ATG4B, MFN2, DRP1, and/or SOD1.

Preferably, said protein comprises at least one protein selected from ATG4B, MFN2, DRP1, and/or SOD1; and wherein the levels of said protein(s) measured in a sample obtained from the subject is used for monitoring the progress or effectiveness of the treatment.

Preferably, said protein comprises at least two proteins selected from ATG4B, MFN2, DRP1, and/or SOD1.

Preferably, said protein comprises at least three proteins selected from ATG4B, MFN2, DRP1, and SOD1.

Preferably, said protein comprises the following four proteins: ATG4B, MFN2, DRP1, and SOD1.

Preferably, said protein is used for monitoring the progress or effectiveness of the long COVID treatment.

Preferably, said protein is for use in the treatment of long COVID, wherein the levels of said protein are measured in a sample obtained from the subject to determine the progress or effectiveness of the long COVID treatment, wherein preferably measuring (a change in) the levels of said protein(s) in said sample compared to a reference is used for monitoring the progress or effectiveness of the long COVID treatment. Preferably said sample is a tissue sample, preferably a biopsy, more preferably a nasal mucosal or bronchial biopsy sample.

Preferably, the subject is a vertebrate subject, preferably a mammalian subject, more preferably a human subject.

The present invention also relates to a kit, which comprises means for measuring the levels of at least one protein, wherein the at least one protein is selected from ATG4B, MFN2, DRP1, and SOD1.

Preferably, the kit comprises means for measuring the levels of at least two proteins selected from ATG4B, MFN2, DRP1, and/or SOD1.

Preferably, the kit comprises means for measuring the levels of at least three proteins selected from ATG4B, MFN2, DRP1, and SOD1.

Preferably, the kit comprises means for measuring the levels of the following four proteins: ATG4B, MFN2, DRP1, and SOD1.

Preferably, the kit comprises antibodies specific for at least one protein, wherein the at least one protein is selected from ATG4B, MFN2, DRP1, and SOD1.

Preferably, the means and/or antibodies are for measuring the levels of at least one protein selected from ATG4B, MFN2, DRP1, and/or SOD1 in a biological sample. More preferably, said biological sample is a tissue sample, more preferably a biopsy sample, even more preferably a nasal mucosal or bronchial biopsy sample.

### Agent Facilitating Mitochondrial Regeneration for Use in the Treatment of Long COVID in a Subject.

Preferably, said agent is an agent that directly or indirectly decreases the levels of at least one protein in the subject, wherein the at least one protein is selected from ATG4B, MFN2, DRP1, and/or SOD1.

Preferably, said agent is selected from coenzyme Q10 (also called CoQ10 or Q10), rapamycin, metformin, mdivi-1 (mitochondrial division inhibitor, a quinazolinone derivative), P110 (a Drp1-derived peptide), thiazolidinediones, pioglitazone, thiamine, idebenone, imeglimin, bezafibrate, epicatechin, alpha-lipoic acid, resveratrol, riboflavin, dichloroacetate, DRP1 modulators, MFN2 modulators, ATG4B modulators, Q1067, MitoQ (NCT05373043), and/or nicotinamide riboside (NCT05703074). More preferably, said agent is coenzyme Q10 (CoQ10 or Q10), rapamycin, and/or metformin.

Preferably, the subject is a vertebrate subject, preferably a mammalian subject, more preferably a human subject.

The present invention also related to a method of treating long COVID in a subject, wherein said method comprises administering to the subject an agent facilitating mitochondrial regeneration or repairing mitochondrial function.

Preferably, said agent is an agent that directly or indirectly decreases the levels of at least one protein in the subject, wherein the at least one protein is selected from ATG4B, MFN2, DRP1, and/or SOD1.

Preferably, the method of treatment comprises administering to a subject an agent selected from coenzyme Q10 (also called CoQ10 or Q10), rapamycin, metformin, mdivi-1 (mitochondrial division inhibitor, a quinazolinone derivative), P110 (a Drp1-derived peptide), thiazolidinediones, pioglitazone, thiamine, idebenone, imeglimin, bezafibrate, epicatechin, alpha-lipoic acid, resveratrol, riboflavin, dichloroacetate, DRP1 modulators, MFN2 modulators, ATG4B modulators, Q1067, MitoQ (NCT05373043), and/or nicotinamide riboside (NCT05703074).

Preferably, the subject is a vertebrate subject, preferably a mammalian subject, more preferably a human subject.

The present invention also relates to a method for diagnosing and treating long COVID in a subject, wherein said method comprises the following steps:
a) providing a biological sample obtained from the subject,
b) measuring the levels of at least one protein in said sample,
   wherein the at least one protein is selected from ATG4B, MFN2, DRP1, and/or SOD1
c) comparing the levels of the at least one protein measured in step b) with a reference, wherein an increase in the levels of the at least one protein in said sample relative to the reference is indicative of Long COVID diagnosis,
d) administering an agent facilitating mitochondrial regeneration or repairing mitochondrial function to a subject diagnosed with long COVID.

Preferably, the at least one protein is as defined above.

Preferably, the biological sample is a tissue sample, preferably a biopsy, more preferably a nasal mucosal or bronchial biopsy sample.

Preferably, the reference is the levels of the at least one protein measured in sample(s) obtained from a healthy subject or a group of healthy subjects, i.e. a subject or a group of subjects not suffering from long COVID.

Preferably, the subject is a vertebrate subject, preferably a mammalian subject, more preferably a human subject.

Preferably, said agent is an agent that directly or indirectly decreases the levels of at least one protein in the subject, wherein the at least one protein is selected from ATG4B, MFN2, DRP1, and SOD1.

Preferably, the method of treatment comprises administering to a subject an agent selected from coenzyme Q10 (also called CoQ10 or Q10), rapamycin, metformin, mdivi-1 (mitochondrial division inhibitor, a quinazolinone derivative), P110 (a Drp1-derived peptide), thiazolidinediones, pioglitazone, thiamine, idebenone, imeglimin, bezafibrate, epicatechin, alpha-lipoic acid, resveratrol, riboflavin, dichloroacetate, DRP1 modulators, MFN2 modulators, ATG4B modulators, Q1067, MitoQ (NCT05373043), and/or nicotinamide riboside (NCT05703074).

### Definitions

The term “Long COVID”, also called “post-COVID-19 syndrome” or “Post-Acute Sequelae of severe acute respiratory syndrome coronavirus 2 (SARS-CoV-2) infection (PASC)” refers to a condition affecting individuals with a suspected or confirmed SARS-CoV-2 infection who experience lasting symptoms for several weeks or months, e.g. a minimum of two months, after the initial infection, and where these symptoms cannot be attributed to another underlying medical condition. The symptoms include fatigue, chronic fatigue, dyspnea, thoracic disorders, joint disorders (e.g. joint pain), sleep problems, mood disorders such as depression and anxiety, headaches, dizziness, impaired memory, cognitive issues commonly referred to as “brain fog”, cardiac symptoms (e.g. cardiac arrhythmias, tachycardia), paresthesia, disorders of smell and taste (in particular anosmia, hyposmia, dysosmia, ageusia, hypogeusia, and dysgeusia), and urticaria and other dermatological issues. Thus, long COVID, as used herein, includes post-COVID-19 syndrome and PASC, as well.

The term “marker” or “biomarker” refers to an indicator, including biological molecules (i.e., proteins, lipids, nucleic acids, sugars, and so forth) which could provide diagnosis or prognosis for a given condition.

The term “mitochondrial DNA” refers to the genetic material found inside the cellular organelle mitochondrium. The size of the mitochondrial DNA is about 16500 base pairs. The “mitochondrial DNA” also encompasses any segments of the mitochondrial genetic material.

The term “circulating cell-free mitochondrial DNA” (also called circulating mitochondrial DNA or cell-free mitochondrial DNA) refers to mitochondrial DNA molecules derived and released from cells into the systemic circulation.

The term “subject” as used herein shall refer to a vertebrate, preferably a homeothermic (mammalian or avian, preferably mammalian) subject, particularly a human being.

The term “biological sample” refers to any biological material in which biomarkers can be found. A biological sample can be a cell, a group or aggregate of cells, a cell culture, a tissue sample (e.g. a biopsy), a biological fluid (such as blood, serum, plasma, cerebrospinal fluid) or an organ. The biological sample is preferably a biological fluid or a tissue sample.

A “reference” or “reference value” as used herein refers to a quantity (e.g. numerical value or level) or other characteristics (e.g. morphology, structural feature) of a biomarker characterizing a subject or group of subjects, which serves as a reference for comparison for subjects for whom in vitro diagnosis of long COVID or treatment of long COVID is to be carried out. The “reference” or “reference value” may refer to a quantity or other characteristics of a given biomarker that characterizes a healthy subject or a group of healthy subjects, i.e. a subject or a group of subjects not suffering from long COVID.

“Comparing” two quantified biomarkers (e.g. levels of biomarkers, concentration of biomarkers, etc.), is to be understood herein to include a comparison of quantities expressed in numerical values characterizing said biomarkers to establish which is higher or lower, or establishing a difference or establishing a ratio of the levels, or values derived from the levels, optionally completed with other mathematical procedures as the quantification or calculation method requires.

The term “treatment” refers to any process, action, application, therapy, or the like, wherein the subject or patient is under aid, in particular medical or veterinarian aid, with the object of improving the subject's or patient's condition, either directly or indirectly.

A “therapy” is understood herein as a method for treatment in which a given medicament or pharmaceutical composition is administered to said patient, preferably administered for a certain period of time with the object of improving the subject's or patient's condition.

An “agent facilitating mitochondrial regeneration” or “mitochondrial regeneration agent” refers to any substance (including drugs, vitamins, cofactors, antioxidants) which helps restore mitochondrial function. Such agents include for example coenzyme Q10 (also called CoQ10 or Q10), rapamycin, metformin, mdivi-1 (mitochondrial division inhibitor, a quinazolinone derivative), P110 (a Drp1-derived peptide), thiazolidinediones, pioglitazone, thiamine, idebenone, imeglimin, bezafibrate, epicatechin, alpha-lipoic acid, resveratrol, riboflavin, dichloroacetate, DRP1 modulators, MFN2 modulators, ATG4B modulators, Q1067, MitoQ (NCT05373043), nicotinamide riboside (NCT05703074), etc.

The term “comprise(s)” or “comprising” or “including” are to be construed herein as having a non-exhaustive meaning and to allow the addition or involvement of further features or method steps or components to anything which comprises the listed features or method steps or components. Such terms can be limited to “consisting essentially of” or “comprising substantially” which is to be understood as consisting of mandatory features or method steps or components listed in a list, e.g. in a claim, whereas allowing to contain additionally other features or method steps or components which do not materially affect the essential characteristics of the use, method, composition or other subject matter.

As used in this specification and the appended claims, the singular forms “a”, “an” and “the” include plural references, and should be construed as including the meaning “one or more”, unless the content clearly dictates otherwise. In general, it is also to be understood that the terminology used herein is for the purpose of describing particular embodiments only and is not intended to be limiting.

### Abbreviations

ATG4B Autophagy Related 4B Cysteine Peptidase
bp base pair
ccf-mtDNA circulating cell-free mitochondrial DNA
COVID-19 Coronavirus Disease 2019
DAMP damage-associated molecular pattern
DRP1 Dynamin-related Protein 1
FIS1 Mitochondrial fission 1 protein
LDH Lactate dehydrogenase
MFN1 Mitofusin 1
MFN2 Mitofusin 2
PASC Post-Acute Sequelae of SARS-CoV-2 infection
PC post-COVID-19
ROS reactive oxygen species
SARS-CoV-2 severe acute respiratory syndrome coronavirus 2
SOD1 Superoxide dismutase 1
TEM transmission electron microscopy

### BRIEF DESCRIPTION OF THE FIGURES

FIG. 1: Analysis of mitochondrial morphology and expression of specific proteins related to mitochondrial function in patients with post-COVID-19 (PC) syndrome and control participants by TEM. Mitochondrial morphology and immunodetection of proteins associated with mitochondrial function in patients (first row, left panel, denoted by ‘PC’) and control (denoted by ‘C’) participants (first row, right panel). Protein markers analyzed: ATG4B. In the second row, violin plots quantitatively present the immunodetection results corresponding to the protein markers listed in the first row of each panel, respectively. Statistical significance between PC and C samples is denoted by asterisks: *p<0.05, ***p<0.001. “ns” indicates no significant differences (p>0.05).

FIG. 2A-2C: Analysis of mitochondrial morphology and expression of specific proteins related to mitochondrial function in patients with post-COVID-19 (PC) syndrome and control participants by TEM. Mitochondrial morphology and immunodetection of proteins associated with mitochondrial function in patients (first row, left panel, denoted by ‘PC’) and control (denoted by ‘C’) participants (first row, right panel. Protein markers analyzed: (2A) SOD1, (2B) DRP1, and (2C) MFN2. In the second row in each of FIG. 2A-2C, violin plots quantitatively present the immunodetection results corresponding to the protein markers listed in the first row of each panel, respectively. Statistical significance between PC and C samples is denoted by asterisks: *p<0.05, ***p<0.001. “ns” indicates no significant differences (p>0.05).

FIG. 3A-3C: Analysis of mitochondrial morphology and expression of specific proteins related to mitochondrial function in patients with post-COVID-19 (PC) syndrome and control participants by TEM. Mitochondrial morphology and immunodetection of proteins associated with mitochondrial function in patients (first row, left panel, denoted by ‘PC’) and control (denoted by ‘C’) participants (first row, right panel. Protein markers analyzed: (3A) LDH, (3B) MFN1, and (3C) FIS1. In the second row in each of FIG. 3A-3C, violin plots quantitatively present the immunodetection results corresponding to the protein markers listed in the first row of each panel, respectively. Statistical significance between PC and C samples is denoted by asterisks: *p<0.05, ***p<0.001. “ns” indicates no significant differences (p>0.05).

FIG. 4: Quantitatively presents the analysis of mitochondrial morphology and copy number differences in PC patients.

FIG. 5: Quantitative analysis of ccf-mtDNA content in patients with post-COVID-19 (PC) syndrome and control participants. Heatmap displaying the levels of ccf-mtDNA for five mitochondrial genes (MTATP6, MTCYTB, MTND1, MTND4, MTND5) in post-COVID-19 (denoted by ‘PC’, vertical stripes) and control (denoted by ‘C’, diagonal stripes) individuals.

FIG. 6A-6F: Violin plots (top panel in each of the subpanels, depicted in different pages), showing the distribution of ccf-mtDNA levels for each mitochondrial gene, alongside Receiver Operating Characteristic (ROC) curves (bottom panel in each of the subpanels, depicted in different pages,) which evaluate the diagnostic potential of ccf-mtDNA measurements in distinguishing between the PC and C groups.

FIG. 7: Mechanisms and consequences of mitochondrial damage and dysfunction in the pathogenesis of Long COVID. This schematic illustrates the cascade of events leading from initial SARS-CoV-2 infection to persistent mitochondrial dysfunction and its systemic effects. The diagram highlights key steps: (1) Initial mitochondrial damage through direct viral interaction and immune-mediated responses; (2) Activation of mitophagy in an attempt to clear damaged mitochondria; (3) Persistent mitochondrial dysfunction due to incomplete removal of damaged mitochondria, evidenced by reduced ccf-mtDNA levels; (4) Resultant systemic effects contributing to the symptomatology of Long COVID; (5) Utilization of ccf-mtDNA as a diagnostic and monitoring tool to assess the extent of mitochondrial dysfunction. Each component integrates findings from our current work, emphasizing the role of mitochondrial damage in the pathogenesis of Long COVID.

### DETAILED DESCRIPTION OF THE INVENTION

Coronavirus Disease 2019 (COVID-19) can lead to severe acute respiratory syndrome, and while most individuals recover within weeks, approximately 30-40% of them experience persistent symptoms collectively known as Long COVID, post-COVID-19 syndrome, or Post-Acute Sequelae of severe acute respiratory syndrome coronavirus 2 (SARS-CoV-2) infection (PASC). These enduring symptoms, including fatigue, respiratory difficulties, body pain, short-term memory loss, concentration issues, and sleep disturbances, can persist for months. According to recent studies, SARS-CoV-2 infection causes prolonged disruptions in mitochondrial function, significantly altering cellular energy metabolism.

A primary goal according to the present invention was to investigate novel biomarkers of mitochondrial dysfunction in Long COVID patients and their correlation with persistent symptoms, particularly chronic fatigue. To achieve this, the present inventors conducted a series of comparative analyses between post-COVID-19 patients and controls. Utilizing transmission electron microscopy, we inspected nasal mucosal and bronchial biopsy samples to identify and characterize mitochondrial structural abnormalities and their association with Long COVID symptoms. The present inventors quantified the levels of proteins crucial to mitochondrial dynamics—specifically Autophagy Related 4B Cysteine Peptidase (ATG4B), Mitofusin 2 (MFN2), and Dynamin-related Protein 1 (DRP1). Elevated levels of these proteins might indicate ongoing mitochondrial dysfunction or compensatory responses within affected cells. Additionally, measuring Superoxide dismutase 1 (SOD1) protein levels provided insights into the oxidative stress status of these patients. By assessing the circulating cell-free mitochondrial DNA (ccf-mtDNA) in blood plasma, we evaluated the integrity and functionality of mitochondrial recycling processes in post-COVID-19 patients. Through these objectives, the present inventors' work sought to validate the hypothesis that persistent mitochondrial dysfunction significantly contributes to the chronic symptoms of Long COVID.

The present inventors' research employed transmission electron microscopy to reveal distinct mitochondrial structural abnormalities in Long COVID patients, notably including significant swelling, disrupted cristae, and an overall irregular morphology, which collectively indicates severe mitochondrial distress. The present inventors noted increased levels of superoxide dismutase 1 which signals oxidative stress, and elevated Autophagy Related 4B Cysteine Peptidase levels, indicating disruptions in mitophagy. Importantly, the present inventors' analysis also identified reduced levels of circulating cell-free mitochondrial DNA (ccf-mtDNA) in these patients, serving as a novel biomarker for the condition.

These findings underscore the crucial role of persistent mitochondrial dysfunction in the pathogenesis of Long COVID. Further exploration of the cellular and molecular mechanisms underlying post-viral mitochondrial dysfunction is critical, particularly to understand the roles of autoimmune reactions and the reactivation of latent viruses in perpetuating these conditions. This comprehensive understanding could pave the way for targeted therapeutic interventions designed to alleviate the chronic impacts of Long COVID. By utilizing circulating ccf-mtDNA and other novel mitochondrial biomarkers, the present invention can enhance diagnostic capabilities and improve the management of this complex syndrome.

The present inventors' work aimed to elucidate the role of mitochondrial dysfunction in Long COVID by examining mitochondrial structure, dynamics, and DNA content in PC patients compared to healthy controls. The present inventors' findings reveal significant mitochondrial abnormalities in PC patients, including compromised mitochondrial integrity, an imbalance in proteins that regulate mitochondrial fusion and fission, and reduced ccf-mtDNA content. Notably, the altered levels of assessed mitochondrial biomarkers in PC patients suggest mitochondrial malfunction and disrupted mitochondrial dynamics, potentially underpinning the persistence of post-COVID symptoms.

Mitochondria are versatile cellular organelles that play a central role in numerous biochemical pathways, including ATP production and fatty acid synthesis, calcium signaling, cell cycle regulation, apoptosis, and innate immune response [57]. The observed mitochondrial structural changes in PC patients, such as dilated cristae and enlarged mitochondria, indicate severe mitochondrial distress. These alterations can impact mitochondrial efficiency, leading to insufficient ATP production and an increase in reactive oxygen species (ROS). The link between such structural abnormalities and the elevated levels of SOD1 underscores a heightened oxidative stress response in PC patients, a condition that can exacerbate cellular damage and prolong recovery from viral infections. The imbalance in mitochondrial dynamics highlighted by increased levels of MFN2 and DRP1 could be indicative of the cell's attempt to maintain mitochondrial function by enhancing fusion and fission processes. However, these compensatory mechanisms may not suffice to restore normal mitochondrial function and could instead lead to further dysregulation of cellular energy metabolism. This dysregulation is critical in understanding the widespread energy deficiency experienced by PC patients, manifesting as chronic fatigue and muscular weakness. Accordingly, research has revealed impairments in mitochondrial respiration, bioenergetics, and gene expression within peripheral blood mononuclear cells of Long COVID patients [58-62]. These deficits suggest that diminished mitochondrial energy production may contribute to prevalent symptoms like fatigue and muscle weakness. Additionally, magnetic resonance spectroscopy has detected mitochondrial dysfunction in the muscle tissue and brains of those affected, supporting clinical observations of exercise intolerance and post-exertional malaise [63-67]. Additional support for the role of mitochondria in Long COVID is provided by biomarker studies. These studies have identified specific markers that indicate mitochondrial dysfunction, further linking it to the condition's persistent symptoms. Elevated levels of circulating biomarkers indicative of oxidative stress and mitochondrial damage, such as F2-isoprostanes and malondialdehyde, PARylation along with decreased levels of antioxidants such as coenzyme Q10, have been documented in Long COVID patients [46, 48 68-73]. These biomarkers underscore the role of oxidative stress in exacerbating mitochondrial dysfunction associated with Long COVID. The significant reduction in circulating ccf-mtDNA levels among PC patients suggests an impaired mitochondrial recycling process. This finding is crucial as it points to a potential systemic impact of mitochondrial dysfunction, which could extend beyond the initially infected cells to affect various tissues and organ systems. The diagnostic potential of ccf-mtDNA underscores its utility in identifying patients with Long COVID, where mitochondrial damage and dysfunction are pivotal to the condition's pathogenesis.

The mechanisms by which SARS-CoV-2 induces mitochondrial damage are likely multifaceted. Direct interactions between viral proteins and mitochondrial components disrupt the normal function and dynamics of mitochondria [74-75] and cause structural damage [44, 76-79]. It has become evident that viruses employ various mechanisms to target host cell mitochondria to support viral particles' growth and survival, further weakening the host's cellular immune response and enhancing cell death. Viral infection often results in the release of damage-associated molecular patterns (DAMPs) that activate the antiviral immune response [80]. mtDNAs belong to mitochondrial DAMPs which are released by damaged cells [81]contributing to heightened state of systemic inflammation [81]. Additionally, it has been reported that SARS-CoV-2 infection increases ROS production, causing oxidative damage to mtDNA and proteins, further exacerbating mitochondrial dysfunction [48]. Indirectly, the inflammatory response and immune dysregulation triggered by the infection can exacerbate mitochondrial damage. These mechanisms together suggest that SARS-CoV-2 not only targets mitochondrial health directly but also creates a systemic environment that perpetuates mitochondrial and cellular dysfunction.

Mitochondria undergo coordinated fusion and fission cycles, leading to transient morphological adaptations essential for various molecular processes such as cell cycle control, immune function, mitochondrial quality control, and apoptosis [82]. Our results suggest that mitochondrial dysfunction in PC patients is associated with disruptions in pathways that regulate mitochondrial fusion-fission and mitophagy. These disorders can exacerbate metabolic imbalance, contributing to post-COVID-19 symptoms [83]. Notably, the mitochondrial dysfunction observed in Long COVID shares similarities with other post-viral syndromes such as Myalgic Encephalomyelitis/Chronic Fatigue Syndrome (ME/CFS) [60, 84-87]. Drawing parallels between these conditions may illuminate common mechanisms and shared therapeutic targets, providing a broader context for understanding post-viral conditions.

The development of autoimmunity following COVID-19 [88-96], wherein the immune system mistakenly targets mitochondrial proteins [97] and other cellular components, could further exacerbate mitochondrial dysfunction [98]. This autoimmune response may contribute to the chronic persistence of symptoms such as fatigue, muscle weakness, and neurological impairments by continually undermining mitochondrial function and preventing recovery.

Moreover, the stress of the infection and subsequent immune system alterations may reactivate latent herpesviruses such as cytomegalovirus (CMV), Epstein-Barr virus (EBV), and human herpesvirus 6 (HHV-6) [99-114], all known to influence mitochondrial function. The reactivation of these viruses during or after COVID-19 can exacerbate mitochondrial damage, thereby contributing to the severity and persistence of Long COVID symptoms [99, 115], further complicating the clinical picture and potentially hindering recovery.

Mitochondrial dysfunction impacts various organs differently, which helps explain the wide range of symptoms associated with Long COVID. In the brain, it may contribute to neurological symptoms like ‘brain fog’ and fatigue. In the heart, it can lead to energy deficits that manifest as cardiac symptoms such as arrhythmias. Additionally, the importance of mitochondria in vascular endothelial function cannot be overlooked [116-120], especially considering that SARS-CoV-2 exhibits endothelial trophism [17]. There is a growing body of literature suggesting that endothelial dysfunction plays a central role in the pathogenesis of both acute COVID-19 and Long COVID. The endothelium relies heavily on mitochondrial integrity for the regulation of vascular tone and maintenance of the blood-brain barrier [116-120]. Mitochondrial dysfunction in endothelial cells can lead to impaired production of nitric oxide, a critical vasodilator, thereby contributing to vascular stiffness, hypertension and impaired blood flow to the brain, muscles and heart. Moreover, endothelial mitochondrial damage might enhance the permeability of the blood-brain barrier, facilitating the influx of inflammatory mediators into the central nervous system. The resulting heightened inflammatory state in the brain can exacerbate neurological symptoms and may also contribute to the multisystem involvement seen in Long COVID. Thus, in Long COVID, mitochondrial dysfunction in the vasculature likely contributes to a range of manifestations, from vasodilator dysfunction to blood-brain barrier disruption. Additionally, immune responses triggered by factors released from damaged mitochondria may contribute to persisting inflammation and thereby to the development of post-COVID-19 conditions [121-123]. These effects collectively compound the complex symptomatology of Long COVID, linking systemic mitochondrial impairment with organ-specific clinical outcomes. The systemic nature of mitochondrial dysfunction thus serves as a unifying pathophysiological mechanism underlying the diverse and persistent symptoms observed in patients with Long COVID.

The insights gained from the present inventors' work pave the way for exploring mitochondrial-targeted therapies as potential treatments for Long COVID [36]. Interventions that enhance mitochondrial function, including the use of mitochondrial-targeted antioxidants, lifestyle modifications like improved diet and exercise, and potentially pharmaceutical interventions, are under investigation [36]. These strategies aim to restore mitochondrial health [48, 49], which could alleviate the broad spectrum of Long COVID symptoms. Among them, several compounds with known mitochondrial protective effects, such as Q1067, MitoQ (NCT05373043), alpha-lipoic acid, nicotinamide riboside (NCT05703074), and resveratrol (NCT05601180) are currently under investigation in clinical trials [124-126]. Further research is needed to explore these therapeutic avenues and to validate the effectiveness of novel biomarker for monitoring disease progression and treatment response.

In particular, identifying reliable biomarkers of mitochondrial dysfunction is critical [36]. In the present inventors' work in accordance with the present invention, the present inventors investigated the utility of plasma mtDNA content as a diagnostic tool for post-COVID-19 conditions. Surprisingly, in contrast to the present inventors' initial hypothesis that increased mitophagy would elevate ccf-mtDNA levels in patients with chronic symptoms, the present inventors observed lower ccf-mtDNA levels. This suggests that while mitochondrial clearance mechanisms are activated, they fail to completely remove damaged mitochondria. Supporting this, the present inventors noted differences in mitochondrial morphology and size between PC patients and controls, indicating persistent mitochondrial abnormalities despite active mitophagy. Importantly, the correlation between reduced ccf-mtDNA levels and symptom severity underscores its potential as a valuable biomarker for diagnosing and monitoring post-COVID-19 conditions, offering a means to differentiate between affected individuals and healthy controls and assess the extent of mitochondrial dysfunction. The development and validation of these and similar biomarkers could significantly improve the diagnosis and monitoring of Long COVID, aiding in the assessment of treatment efficacy and understanding disease progression [36].

In conclusion, the present inventors' work in accordance with the present invention has substantiated the pivotal role of mitochondrial dysfunction in the chronic manifestations of Long COVID [36]. The present invention extends understanding of these underlying mechanisms and consequently it becomes clear that aging may play a significant modulatory role in these processes [17]. Aging is known to induce mitochondrial dysfunction across various cell types, contributing to the functional decline of these organs and rendering cells and mitochondria less resilient. This vulnerability may exacerbate the severity of mitochondrial damage observed in Long COVID, making the elderly particularly susceptible to prolonged and severe post-viral symptoms [17]. Therefore, it is imperative that future studies explore how aging influences mitochondrial dynamics in the context of Long COVID. Such research could provide insights into age-specific therapeutic interventions and preventive measures, ultimately aiding in the development of targeted strategies that not only improve the quality of life for older adults but also reduce the broader, long-term health impacts of the COVID-19 pandemic. By integrating insights from various medical disciplines and drawing parallels with other post-viral syndromes, the management of Long COVID can be enhanced, paving the way for interventions that address the multifaceted aspects of this condition in an age-sensitive manner.

In an embodiment, the at least one protein used in the in vitro diagnostic methods, the at least one protein for use in the treatment of long COVID, the at least one protein used for monitoring a long COVID treatment or used in the method of treatment is selected from Autophagy Related 4B Cysteine Peptidase (ATG4B), Mitofusin 2 (MFN2), Dynamin-related Protein 1 (DRP1), and/or Superoxide dismutase 1 (SOD1).

Preferably, the at least one protein comprises two proteins selected from ATG4B, MFN2, DRP1, and SOD1. Preferably, the at least one protein comprises ATG4B and MFN2. Preferably, the at least one protein comprises ATG4B and DRP1. Preferably, the at least one protein comprises ATG4B and SOD1. Preferably, the at least one protein comprises MFN2 and DRP1. Preferably, the at least one protein comprises MFN2 and SOD1. Preferably, the at least one protein comprises DRP1 and SOD1.

Preferably, the at least one protein comprises three proteins selected from ATG4B, MFN2, DRP1, and SOD1. Preferably, the at least one protein comprises ATG4B, MFN2 and DRP1. Preferably, the at least one protein comprises ATG4B, MFN2 and SOD1. Preferably, the at least one protein comprises ATG4B, DRP1 and SOD1. Preferably, the at least one protein comprises MFN2, DRP1 and SOD1.

Preferably, the at least one protein comprises four proteins, which are the following: ATG4B, MFN2, DRP1, and SOD1.

In an embodiment, the circulating cell-free mitochondrial DNA (ccf-mtDNA) used in the in vitro diagnostic methods, the ccf-mtDNA for use in the treatment of long COVID, the ccf-mtDNA used for monitoring a long COVID treatment or used in the method of treatment is selected from MTATP6-, MTCYTB-, MTND1-, MTND4-, and MTND5-specific DNAs.

Preferably, the ccf-mtDNA comprises two mitochondrial DNA segments or mitochondrial genes selected from MTATP6-, MTCYTB-, MTND1-, MTND4-, and MTND5-specific DNAs. Preferably, the ccf-mtDNA comprises MTATP6- and MTCYTB-specific DNA. Preferably, the ccf-mtDNA comprises MTATP6- and MTND1-specific DNA. Preferably, the ccf-mtDNA comprises MTATP6- and MTND4-specific DNA. Preferably, the ccf-mtDNA comprises MTATP6- and MTND5-specific DNA. Preferably, the ccf-mtDNA comprises MTCYTB- and MTND1-specific DNA. Preferably, the ccf-mtDNA comprises MTCYTB- and MTND4-specific DNA. Preferably, the ccf-mtDNA comprises MTCYTB- and MTND5-specific DNA. Preferably, the ccf-mtDNA comprises MTND1- and MTND4-specific DNA. Preferably, the ccf-mtDNA comprises MTND1- and MTND5-specific DNA.

Preferably, the ccf-mtDNA comprises MTND4- and MTND5-specific DNA.

Preferably, the ccf-mtDNA comprises three mitochondrial DNA segments or mitochondrial genes selected from MTATP6-, MTCYTB-, MTND1-, MTND4-, and MTND5-specific DNAs. Preferably, the ccf-mtDNA comprises MTATP6-, MTCYTB- and MTND1-specific DNA. Preferably, the ccf-mtDNA comprises MTATP6-, MTCYTB- and MTND4-specific DNA. Preferably, the ccf-mtDNA comprises MTATP6-, MTCYTB- and MTND5-specific DNA. Preferably, the ccf-mtDNA comprises MTATP6-, MTND1- and MTND4-specific DNA. Preferably, the ccf-mtDNA comprises MTATP6-, MTND1- and MTND5-specific DNA. Preferably, the ccf-mtDNA comprises MTATP6-, MTND4- and MTND5-specific DNA. Preferably, the ccf-mtDNA comprises MTCYTB-, MTND1- and MTND4-specific DNA. Preferably, the ccf-mtDNA comprises MTCYTB-, MTND1- and MTND5-specific DNA. Preferably, the ccf-mtDNA comprises MTCYTB-, MTND4- and MTND5-specific DNA. Preferably, the ccf-mtDNA comprises MTND1-, MTND4- and MTND5-specific DNA.

Preferably, the ccf-mtDNA comprises four mitochondrial DNA segments or mitochondrial genes selected from MTATP6-, MTCYTB-, MTND1-, MTND4-, and MTND5-specific DNAs. Preferably, the ccf-mtDNA comprises MTATP6-, MTCYTB-, MTND1-, and MTND4-specific DNAs. Preferably, the ccf-mtDNA comprises MTATP6-, MTCYTB-, MTND1-, and MTND5-specific DNAs. Preferably, the ccf-mtDNA comprises MTATP6-, MTCYTB-, MTND4-, and MTND5-specific DNAs. Preferably, the ccf-mtDNA comprises MTATP6-, MTND1-, MTND4-, and MTND5-specific DNAs. Preferably, the ccf-mtDNA comprises MTCYTB-, MTND1-, MTND4-, and MTND5-specific DNAs.

Preferably, the ccf-mtDNA comprises five mitochondrial DNA segments or mitochondrial genes, which are the following: MTATP6-, MTCYTB-, MTND1-, MTND4-, and MTND5-specific DNAs.

### EXAMPLES

### Example 1: Materials and Methods

### Cohort Characteristics

For the measurement of circulating cell-free mitochondrial DNA (ccf-mtDNA), our study enrolled 32 post-COVID-19 (PC) patients and 31 healthy volunteers, with median ages of 46 and 44 years, respectively. The most prevalent symptoms among PC patients included disorders of smell and taste—specifically anosmia, hyposmia, dysosmia, ageusia, hypogeusia, and dysgeusia. Additionally, these patients frequently reported impaired memory, fatigue, paresthesia, cardiac arrhythmias, tachycardia, dyspnea, as well as thoracic and joint disorders, urticaria, and other dermatological issues (Table 1, left part). The selection of the PC patients was carried out as described by Pavli et al. [50].

For transmission electron microscopy (TEM) analysis, nasal mucosal and bronchial biopsy samples were collected from five PC patients (median age: 28 years) and five controls who exhibited no post-COVID-19 symptoms but were diagnosed with secondary ciliary dyskinesia (median age: 10 years). The primary symptoms of PC patients were smell disorders—anosmia, hyposmia, and dysosmia. Other reported symptoms included taste disorders—ageusia, hypogeusia, and dysgeusia—, fatigue, and various respiratory conditions (Table 1, right part).

TABLE 1
Cohort characteristics for transmission electron microscopy (TEM)
and circulating cell-free mitochondrial DNA (ccf-mtDNA) studies
Cohort characteristics

| | | ccf-mtDNA | | TEM | |
|---|---|---|---|---|---|
| | | PC | C | PC | C |
| Age | Median age (years) | 46 | 44 | 28 | 10 |
| Sex | Female (number of participants) | 24 | 21 | 3 | 1 |
| distribution | Male (number of participants) | 8 | 10 | 2 | 4 |
| Symptoms | Anosmia/Hyposmia/Dysosmia | 16 | — | 5 | — |
| | Ageusia/Hypogeusia/Dysgeusia | 8 | — | 1 | — |
| | Impaired memory | 2 | — | — | — |
| | Fatigue | 2 | — | 1 | — |
| | Paresthesia | 2 | — | — | — |
| | Cardiac arrhytmia | 1 | — | — | — |
| | Tachycardia | 1 | — | — | — |
| | Dyspnea | 1 | — | — | — |
| | Thoracic disorders | 1 | — | — | — |
| | Joint disorders | 1 | — | — | — |
| | Urticaria | 1 | — | — | — |
| | Other respiratory disorder | — | — | 4 | — |
| | Other dermatological condition | 1 | — | — | — |

### Sample Preparation and Post-Embedding for Immunohistochemistry

All cases of human nasal mucosa and bronchial biopsy were previously diagnosed and collected from the archives of the University of Szeged. All specimens were initially preserved in a 3% glutaraldehyde solution supplemented with dextran. Upon arrival at the Department of Pathology, both control (n=5) and PC (n=5) samples underwent a post-fixation in fresh 3% glutaraldehyde solution. The samples were then rinsed in phosphate-buffered saline (PBS) and fixed for 1 hour in 2% osmium tetroxide. The specimens were dehydrated through a graded series of ethanol concentrations, followed by rinsing in uranyl acetate and acetone. Subsequently, they were embedded in Embed812 resin (Electron Microscopy Sciences; Hatfield, PA, USA). Ultrathin sections (70 nm) were prepared using an Ultracut S ultra-microtome (Leica, Wetzlar, Germany) and mounted on copper grids [51].

Post-embedding sections were blocked with 1% bovine serum albumin for 20 minutes, and then washed three times in PBS. They were incubated with primary antibodies at room temperature for either 1 hour or 3 hours, depending on the specific antibody (Table 2). After washing in PBS, sections were incubated with appropriate secondary antibodies—anti-rabbit (for DRP1, MFN2, ATG4B, FIS1, and LDH) or anti-mouse (for MFN1)—for 3 hours at room temperature (Table 3). Finally, sections were counterstained with 0.25% uranyl acetate (Electron Microscopy Sciences, Hatfield, PA, USA) and 3% lead citrate (Leica, Wetzlar, Germany) to enhance contrast [52].

TABLE 2
Primary antibodies used in immunohistochemistry for TEM

| | | | Dilution; | | |
|---|---|---|---|---|---|
| Antibody | Target Protein | Host species | incubation time | Catalog number | Supplier |
| anti-DRP1 | Dynamin-related | rabbit | 1:25; 1 h | ab184247 | Abcam, Cambridge, |
| | protein 1 | | | | UK |
| anti-MFN1 | Mitofusin 1 | mouse | 1:50; 1 h | MA5-36240 | Invitrogen, Waltham, |
| | | | | | Massachusetts, USA |
| anti-MFN2 | Mitofusin 2 | rabbit | 1:25; 3 h | ab219730 | Abcam, Cambridge, |
| | | | | | UK |
| anti-ATG4B | Autophagy- | rabbit | 1:50; 1 h | 710915 | Invitrogen, Waltham, |
| | related protein 4B | | | | Massachusetts, USA |
| anti-FIS1 | Mitochondrial | rabbit | 1:800; 1 h | ab229969 | Abcam, Cambridge, |
| | fission 1 protein | | | | UK |
| anti-SOD1 | Superoxide | mouse | 1:25; 1 h | MA1-105 | Invitrogen, Waltham, |
| | dismutase 1 | | | | Massachusetts, USA |
| anti-LDH | Lactate | rabbit | 1:25; 1 h | ab52488 | Abcam, Cambridge, |
| | dehydrogenase | | | | UK |

TABLE 3
Secondary antibodies used in immunohistochemistry for TEM. Dilutions are
provided by the supplier and optimized for use in TEM to ensure specific
binding and minimal background. Proper handling and storage of antibodies
were ensured as per supplier recommendations to maintain activity.

| Secondary | Host | Size of colloidal | | Catalog | |
|---|---|---|---|---|---|
| antibodies | species | gold particles | Dilution | number | Supplier |
| anti-mouse IgG | goat | 10 nm | 1:20 | G3779 | Sigma-Aldrich, St. Louis, |
| | | | | | MO, USA |
| anti-rabbit IgG | goat | 18 nm | 1:40 | 111-215-144 | Sigma-Aldrich, St. Louis, |
| | | | | | MO, USA |

### Quantification of Immunohistochemistry

For each sample, five cells were imaged using a JEOL JEM 1400 TEM (JEOL; Tokyo, Japan) at magnifications of 12,000× and 20,000×. Images were captured using TEM Center software (JEOL; Tokyo, Japan). To quantify the data, each image was analyzed using the point counting grid method with Image-Pro Plus software (Media Cybernetics, Rockville, Maryland, USA). A 20×20 grid was superimposed over each image, and intersections of grid points with mitochondria were counted. Additionally, the number of gold particles intersected by the grids within mitochondrial regions was tallied. This mitochondrial-associated gold particle count was then normalized to the delimited mitochondrial area for each image.

Due to the non-normal distribution of the data, statistical analysis was performed using the nonparametric Mann-Whitney U test. All statistical evaluations were executed using SPSS software (IBM SPSS Statistics 29; New York, USA). To visually represent the data distribution, violin plots were generated using the Flourish online tool [53].

### Plasma Isolation

Blood samples were collected from PC patients and healthy individuals using 10 ml cell-free DNA BCT tubes (Streck). The tubes were gently inverted ten times to mix and then centrifuged for 10 minutes at 2,000 rpm at 4° C. The upper plasma layer was carefully transferred to a sterile tube and centrifuged again for 10 minutes at 4,500 rpm at 4° C. to eliminate any residual cellular components. Two milliliters of the clarified plasma were then used for each subsequent isolation procedure.

In more detail the protocol for plasma isolation:
1. PAXgene Blood ccfDNA Tube/cell-free DNA BCT tube (Streck) of 10 ml was filled with blood up to about 80-90%;
2. then the tube was immediately inverted, slowly and gently, then this inverting step were repeated 10 times so that the liquid in the tube mixed well with the blood;
3. after that, centrifugation can be started immediately, but if there is no time for this on the same day, the blood can stand for 24 hours at room temperature (the tube should be upright);
4. the centrifugation was performed in a swinging rotor joint (the PAXgene/Streck tubes fit into the rotor suitable for a 15 ml falcon tube) at 2000 rpm, for 10 minutes, at 4° C.;
5. the plasma (i.e., the upper yellowish phase) was then carefully pipetted into a clean, factory-sterile 15 ml falcon tube in such a way that nothing from the lower bloody phase was transferred (it is advised to leave a little of the plasma on the lower phase to make sure that nothing from the bloody phase is transferred)—if the tube was filled with blood as described in point 1 above, about 4 ml of plasma is produced from said about 9-10 ml of blood;
6. then another centrifugation step was carried out to completely remove other cellular debris—this centrifugation was carried out at 4500 rpm, for 10 min, at 4° C.;
7. then the supernatant was carefully pipetted into a clean, factory-sterile 15 ml falcon tube;
8. optionally, the plasma was divided into 1 ml aliquots into freezer tubes and placed at −80° C.;
9. optionally, transportation of the samples can be carried out in two ways:
   a) transportation of the collected blood after inverting within 24 hours at room temperature, or
   b) after step 8, transporting the frozen plasma on dry ice.
      Ccf-DNA Isolation and mtDNA Content Measurement

The QIAamp MinElute ccf-DNA Mini Kit (Qiagen) was employed for the isolation of circulating cell-free DNA (ccf-DNA) following the manufacturer's protocol. The concentration of isolated ccf-DNA was determined using a Qubit 4 fluorometer (Invitrogen). For each quantitative PCR (qPCR) reaction, 0.5 ng of ccf-DNA was used. Relative quantification of mitochondrial DNA (mtDNA) content was performed using qPCR (Rotor-Gene Q, Qiagen) with specific primers, employing cyclophilin B as an internal control to ensure accurate and consistent results. The specific primers are listed in Table 4.

TABLE 4
Primers used in qPCR for relative quantification of
mitochondrial DNA (mtDNA)

| | Forward 5′-3′ primer | Reverse 5′-3′ primer |
|---|---|---|
| MTCYTB | AGCCAACCCCTTAAACACCC | TCATTCGGGCTTGATGTGGG |
| | (SEQ ID NO: 1) | (SEQ ID NO: 2) |
| MTND4 | CCTGACTCCTACCCCTCACA | TGGATAAGTGGCGTTGGCTT |
| | (SEQ ID NO: 3) | (SEQ ID NO: 4) |
| MTATP6 | CCCACTTCTTACCACAAGGCA | TGGGGATAAGGGGTGTAGGT |
| | (SEQ ID NO: 5) | (SEQ ID NO: 6) |
| MTND5 | ACCACATCATCGAAACCGCA | GATAGGGCTCAGGCGTTTGT |
| | (SEQ ID NO: 7) | (SEQ ID NO: 8) |
| MTND1 | AAAGAGCCCCTAAAACCCGC | CGGTGATGTAGAGGGTGATGG |
| | (SEQ ID NO: 9) | (SEQ ID NO: 10) |

### Statistical Analysis of Ccf-mtDNA Content Measurements

To visualize the discriminating potential of the measured ccf-mtDNA, a heat map was generated using the ClustVis online tool [54]. Statistical differences in ccf-mtDNA content between PC patients and healthy volunteers were assessed using independent samples t-tests performed with SPSS software (IBM SPSS Statistics 29; New York, USA). Additionally, violin plots were created using the Flourish online tool to provide a detailed view of the data distribution [53].

To evaluate the diagnostic potential of the ccf-mtDNA measurements, Receiver Operating Characteristic (ROC) curves and the corresponding Area Under the Curve (AUC) values were calculated using SPSS software. These analyses help determine the effectiveness of ccf-mtDNA levels in distinguishing between PC patients and healthy controls.

### Ethics Statement

This study received ethical approval from the Institutional Review Board of the Albert Szent-Györgyi Clinical Centre at the University of Szeged (approval number 100/2022-SZTE RKEB). All procedures performed in studies involving human participants were in accordance with the ethical standards of the institutional and national research committee and with the 1964 Helsinki Declaration and its later amendments.

### Example 2: Results

### Structural and Functional Mitochondrial Impairment in Post-COVID-19 Syndrome

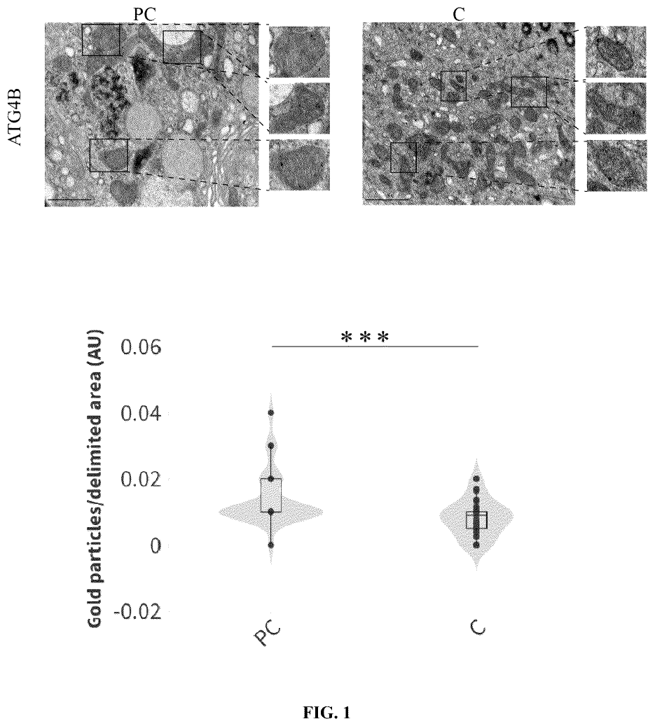

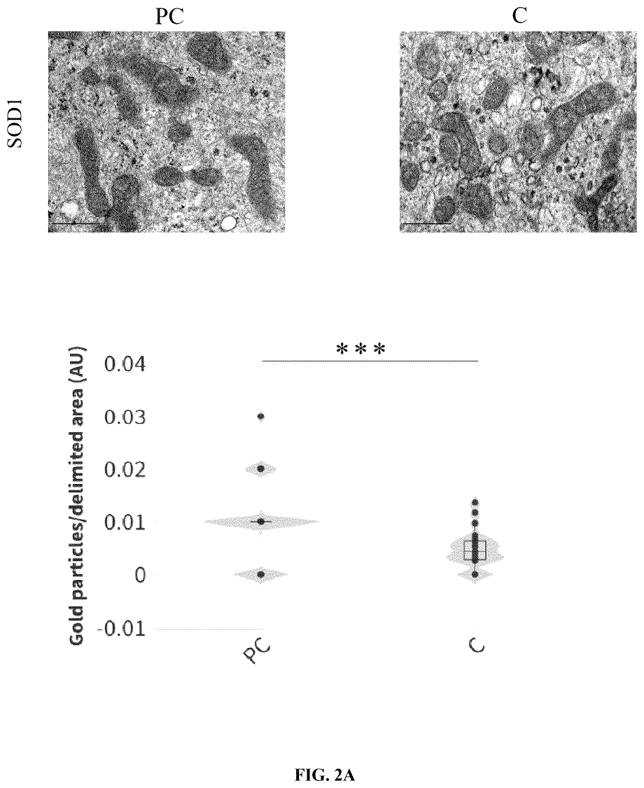

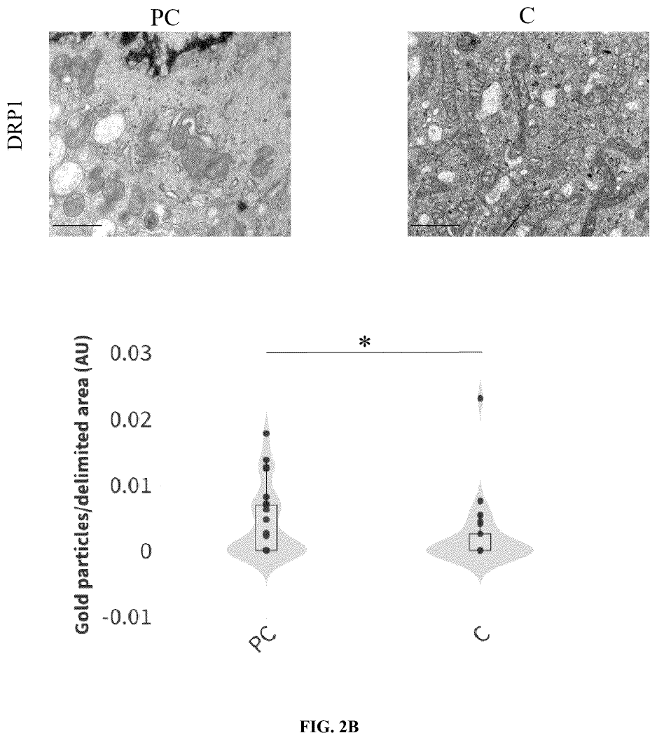

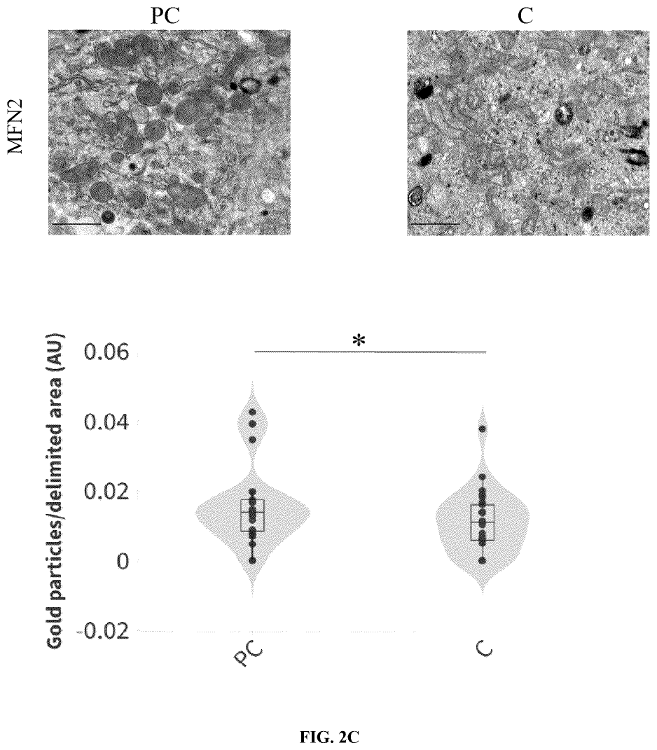

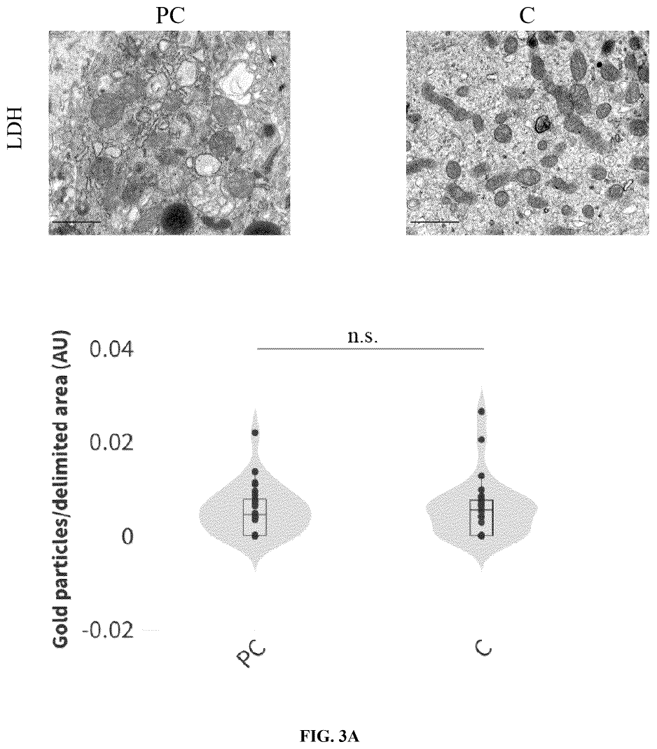

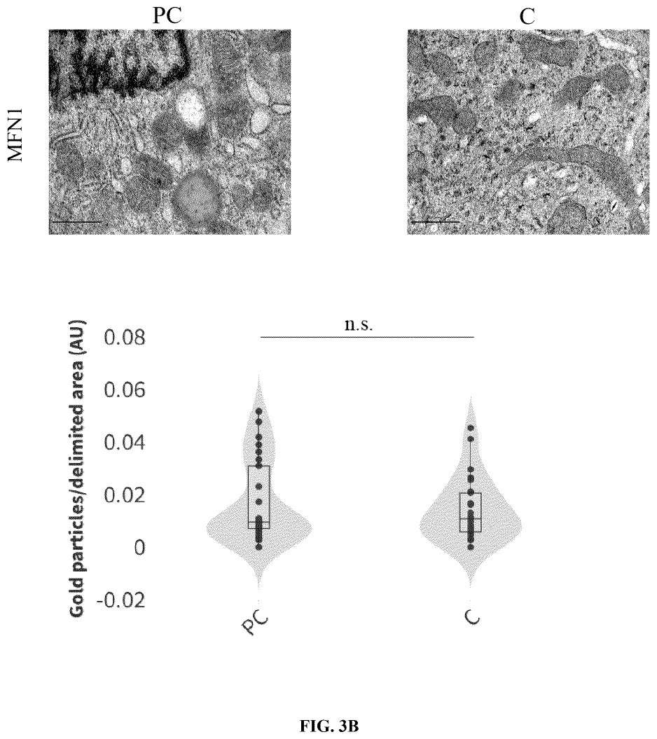

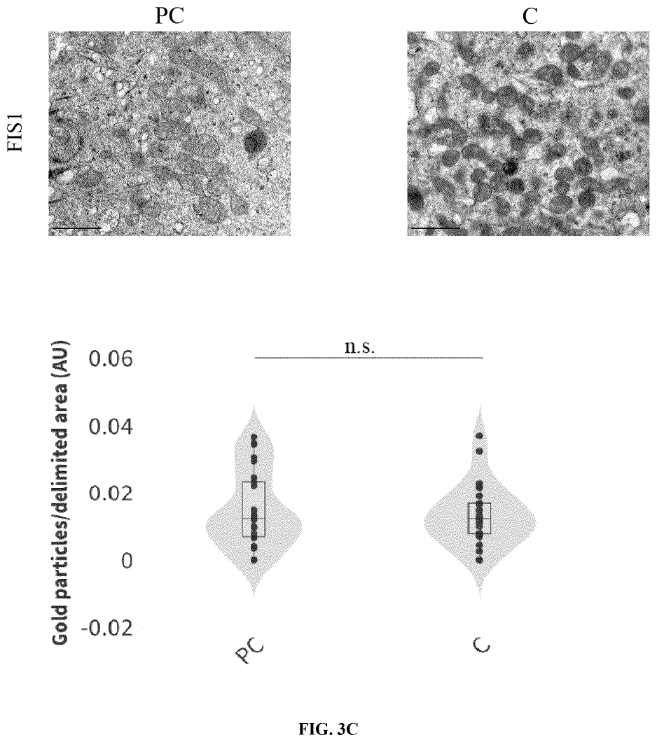

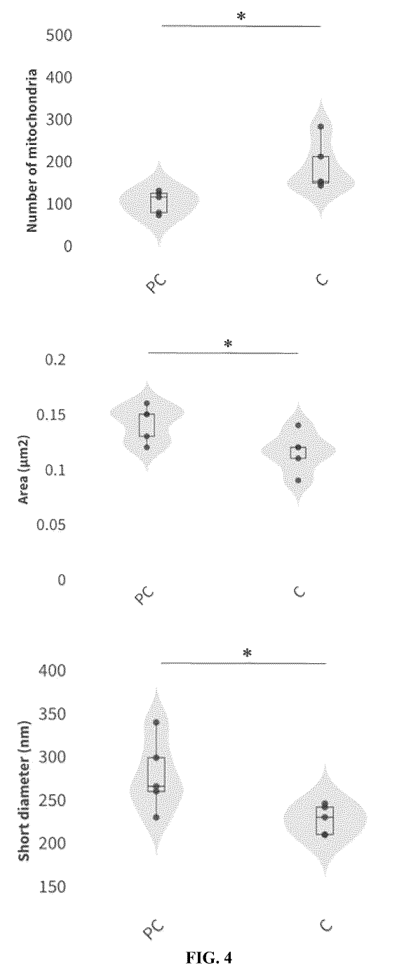

Using TEM, the present inventors examined mitochondrial ultrastructure in nasal mucosal and bronchial needle biopsies from five PC and five control patients. TEM analysis revealed distorted mitochondrial integrity in PC patients, characterized by dilated and washed-out cristae and enlarged mitochondria compared to controls. Additionally, protein levels related to mitochondrial dynamics were quantified. Mitofusin 1 (MFN1) and MFN2 are mitochondrial outer membrane GTPases responsible for mitochondrial outer membrane fusion [55]. Mitochondrial fission 1 protein (FIS1) is involved in mitochondrial fission via DRP1 binding, a fission protein activated by cellular stress and implicated in calcium uptake [56]. While MFN1 and FIS1 levels were comparable to controls, MFN2 and DRP1 levels were elevated, indicating a disrupted balance between mitochondrial fusion and fission (FIG. 2A-2C, FIG. 3A-3C). Despite no observed changes in Lactate dehydrogenase (LDH) levels (FIG. 3A-3C), the morphological changes in mitochondria hinted at underlying mitochondrial damage. Elevated levels of superoxide dismutase 1 (SOD1) in PC patients were consistent with increased reactive oxygen species (ROS) (FIG. 2A-2C). To further investigate mitochondrial recycling, we assessed ATG4B levels, finding them to be higher in PC patients, supporting the hypothesis of enhanced mitophagy as a response to mitochondrial dysfunction (FIG. 1). We also quantified the morphological changes occurring on the mitochondria of the PC patients which revealed severe morphological and mitochondrial number changes in the cells (FIG. 4).

Diminished Circulating Cell-Free mtDNA Content in PC Patients

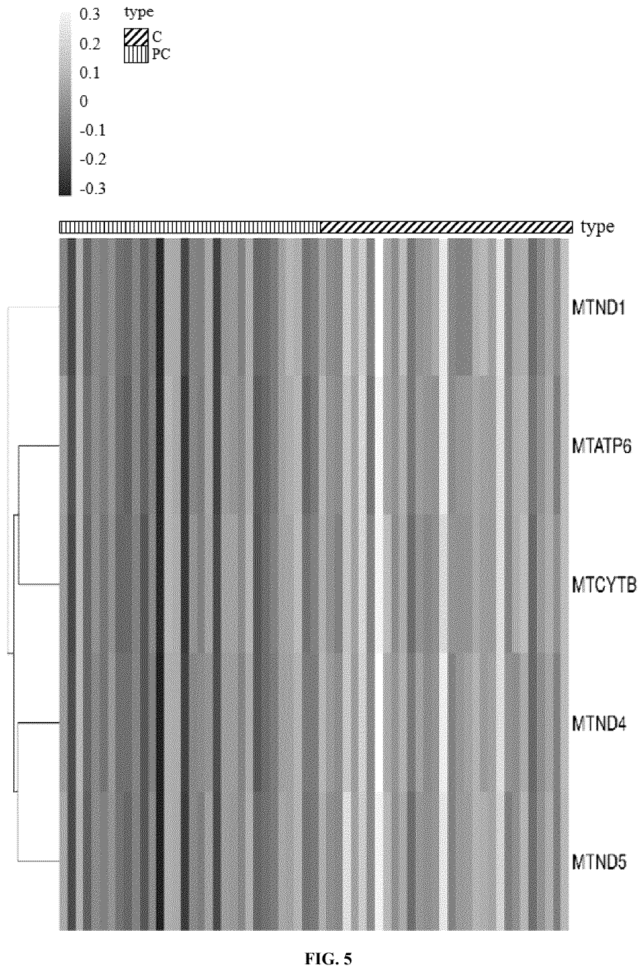

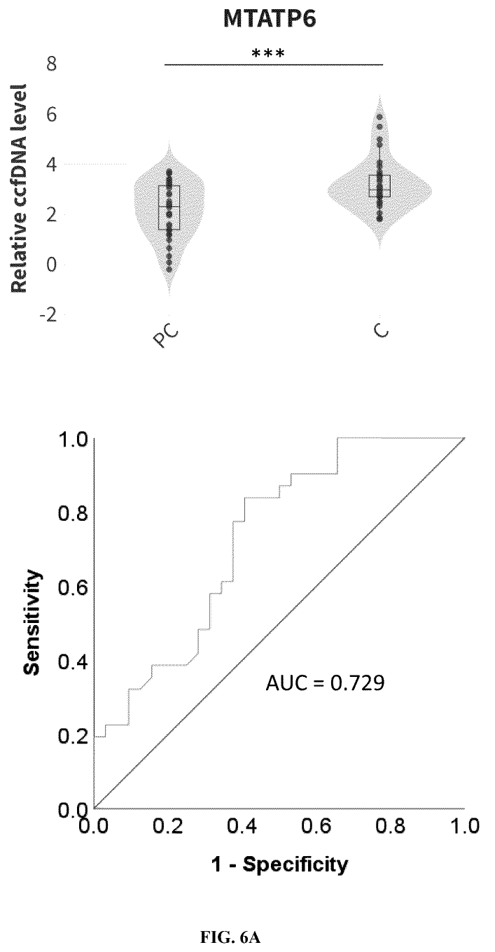

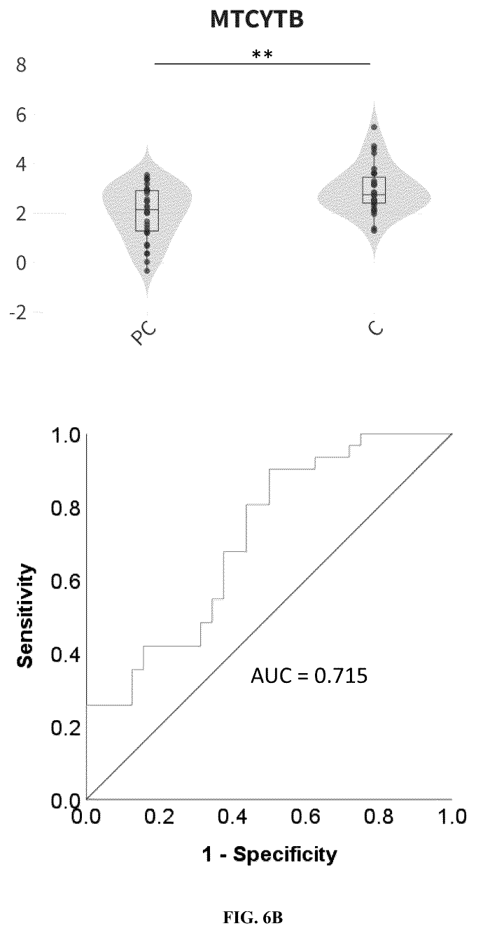

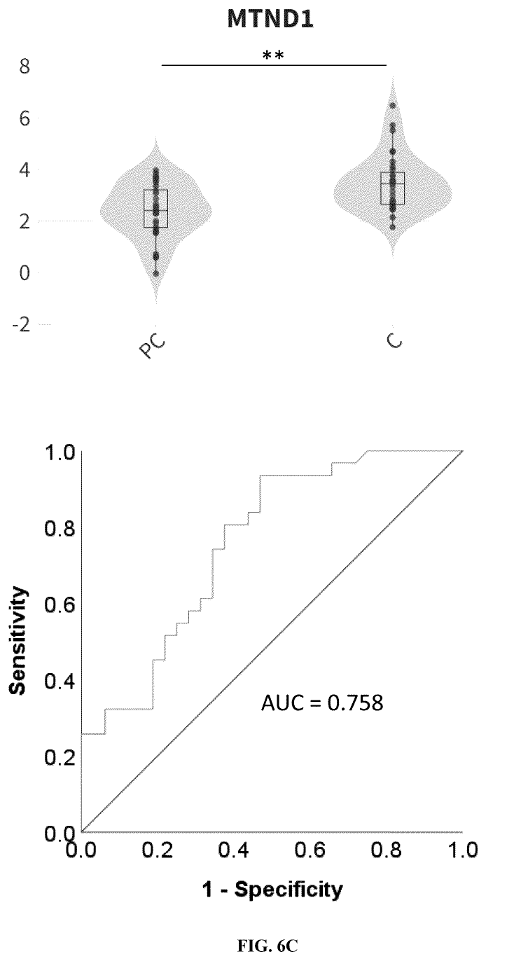

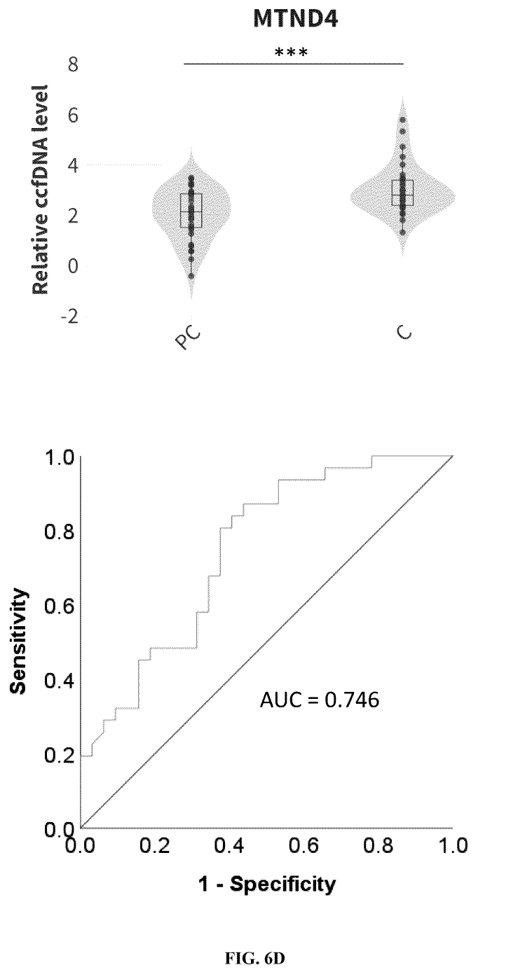

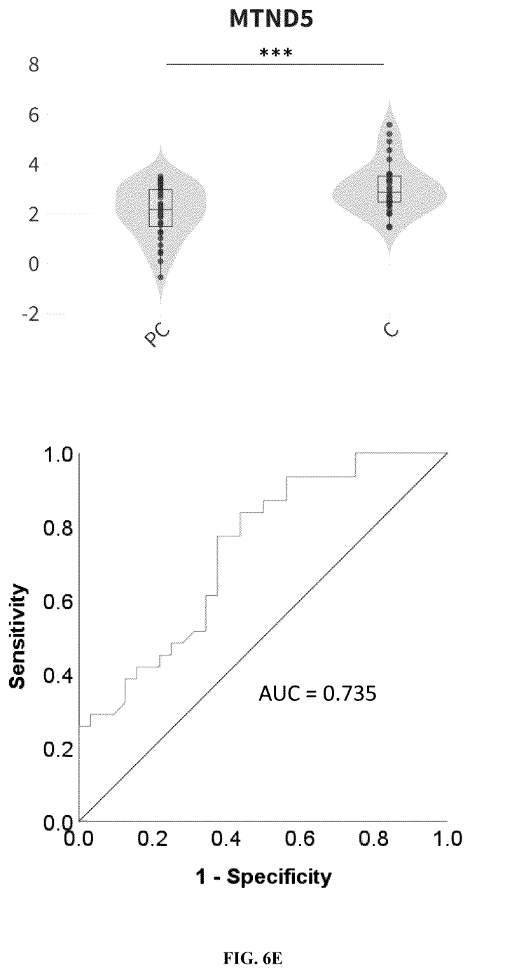

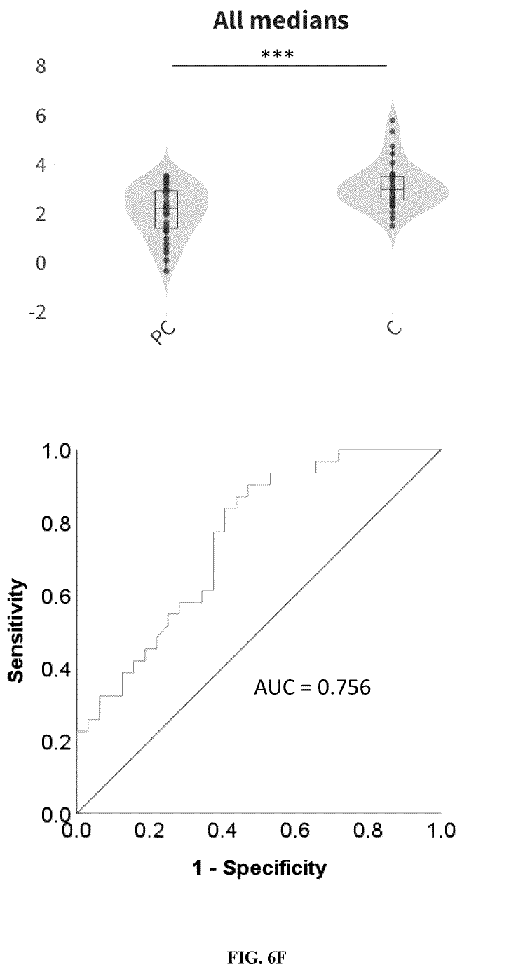

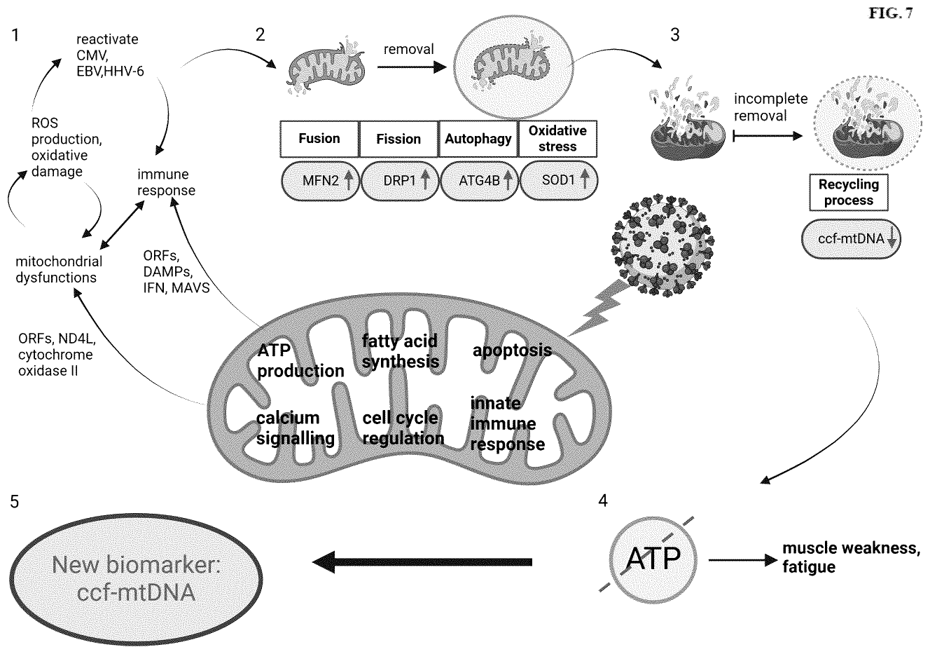

We developed a standardized qPCR method to measure specific mitochondrial DNA (mtDNA) content in the plasma of PC and healthy volunteers. The study included 32 PC and 31 control participants. We quantified MTATP6-, MTCYTB-, MTND1-, MTND4-, and MTND5-specific plasma ccf-mtDNA content. The selection of these genes ensured comprehensive coverage of the mitochondrial genome, providing a robust evaluation of mitochondrial DNA integrity and quantity. Our findings revealed a significant reduction in ccf-mtDNA content in PC patients compared to healthy controls, indicating potential mitochondrial recycling dysfunction (FIG. 5, FIG. 6A-6F). To enhance the robustness of our results, we computed the median values from the individual ccf-mtDNA measurements and consolidated them into a single comprehensive dataset (denoted as “all medians”). This aggregate analysis reaffirmed a substantial reduction in mtDNA levels among PC patients relative to healthy controls. The significance of these observations was further substantiated by statistical analyses, which revealed a consistent pattern of diminished ccf-mtDNA levels across the PC cohort (FIG. 5). The receiver operating characteristic (ROC) curves for each mitochondrial gene region confirmed the diagnostic utility of ccf-mtDNA, with area under the curve (AUC) values ranging from 0.715 to 0.758, suggesting moderate to high accuracy in distinguishing between the two cohorts (FIG. 6A-6F).

### REFERENCES

1. World Health Organization. WHO COVID-19 Dashboard. https://data.who.int/dashboards/covid19/deaths?n=o accessed on 07/08/2024.
2. Nikolich-Zugich J, Knox K S, Rios C T, Natt B, Bhattacharya D and Fain M J. SARS-CoV-2 and COVID-19 in older adults: what we may expect regarding pathogenesis, immune responses, and outcomes. Geroscience. 2020; 42:505-514.
3. Peterfi A, Meszaros A, Szarvas Z, Penzes M, Fekete M, Feher A, Lehoczki A, Csipo T and Fazekas-Pongor V. Comorbidities and increased mortality of COVID-19 among the elderly: A systematic review. Physiol Int. 2022.
4. Fekete M, Szarvas Z, Fazekas-Pongor V, Feher A, Dosa N, Lehoczki A, Tarantini S and Varga J T. COVID-19 infection in patients with chronic obstructive pulmonary disease: From pathophysiology to therapy. Minireview. Physiol Int. 2022.
5. Feher A, Szarvas Z, Lehoczki A, Fekete M and Fazekas-Pongor V. Co-infections in COVID-19 patients and correlation with mortality rate. Minireview. Physiol Int. 2022.
6. Quarleri J, Galvan V and Delpino M V. Omicron variant of the SARS-CoV-2: a quest to define the consequences of its high mutational load. Geroscience. 2022; 44:53-56.
7. Chakraborty C, Bhattacharya M, Sharma A R, Dhama K and Lee S S. Continent-wide evolutionary trends of emerging SARS-CoV-2 variants: dynamic profiles from Alpha to Omicron. Geroscience. 2022:1-22.
8. O'Mahoney L L, Routen A, Gillies C, Ekezie W, Welford A, Zhang A, Karamchandani U, Simms-Williams N, Cassambai S, Ardavani A, Wilkinson T J, Hawthorne G, Curtis F, Kingsnorth A P, Almaghawi A, Ward T, Ayoubkhani D, Banerjee A, Calvert M, Shafran R, Stephenson T, Sterne J, Ward H, Evans R A, Zaccardi F, Wright S and Khunti K. The prevalence and long-term health effects of Long Covid among hospitalised and non-hospitalised populations: A systematic review and meta-analysis. EClinicalMedicine. 2023; 55:101762.
9. WHO. Post COVID-19 condition (Long COVID). https://www.who.int/europe/news-room/fact-sheets/item/post-covid-19-condition. Accessed at 2024.06.02.
10. Bhattacharjee N, Sarkar P and Sarkar T. Beyond the acute illness: Exploring long COVID and its impact on multiple organ systems. Physiol Int. 2023; 110:291-310.
11. Monje M and Iwasaki A. The neurobiology of long COVID. Neuron. 2022; 110:3484-3496.
12. Mansell V, Hall Dykgraaf S, Kidd M and Goodyear-Smith F. Long COVID and older people. Lancet Healthy Longev. 2022; 3:e849-e854.
13. Di Gennaro F, Belati A, Tulone O, Diella L, Fiore Bavaro D, Bonica R, Genna V, Smith L, Trott M, Bruyere O, Mirarchi L, Cusumano C, Dominguez L J, Saracino A, Veronese N and Barbagallo M. Incidence of long COVID-19 in people with previous SARS-Cov2 infection: a systematic review and meta-analysis of 120,970 patients. Intern Emerg Med. 2023; 18:1573-1581.
14. Chen B, Julg B, Mohandas S, Bradfute S B and Force RMPT. Viral persistence, reactivation, and mechanisms of long COVID. Elife. 2023; 12.
15. Saito S, Shahbaz S, Luo X, Osman M, Redmond D, Cohen Tervaert J W, Li L and Elahi S. Metabolomic and immune alterations in long COVID patients with chronic fatigue syndrome. Front Immunol. 2024; 15:1341843.
16. Elizalde-Diaz J P, Miranda-Narvaez C L, Martinez-Lazcano J C and Martinez-Martinez E. The relationship between chronic immune response and neurodegenerative damage in long COVID-19. Front Immunol. 2022; 13:1039427.
17. Russell S J, Parker K, Lehoczki A, Lieberman D, Partha I S, Scott S J, Phillips L R, Fain M J and Nikolich J Z. Post-acute sequelae of SARS-CoV-2 infection (Long COVID) in older adults. Geroscience. 2024.
18. Greenhalgh T, Sivan M, Perlowski A and Nikolich J Z. Long COVID: a clinical update. Lancet. 2024.
19. Chilunga F P, Appelman B, van Vugt M, Kalverda K, Smeele P, van Es J, Wiersinga W J, Rostila M, Prins M, Stronks K, Norredam M and Agyemang C. Differences in incidence, nature of symptoms, and duration of long COVID among hospitalised migrant and non-migrant patients in the Netherlands: a retrospective cohort study. Lancet Reg Health Eur. 2023; 29:100630.
20. Qi C, Osborne T, Bailey R, Cooper A, Hollinghurst J P, Akbari A, Crowder R, Peters H, Law R J, Lewis R, Smith D, Edwards A and Lyons R A. Impact of COVID-19 pandemic on incidence of long-term conditions in Wales: a population data linkage study using primary and secondary care health records. Br J Gen Pract. 2023; 73:e332-e339.
21. Frallonardo L, Segala F V, Chhaganlal K D, Yelshazly M, Novara R, Cotugno S, Guido G, Papagni R, Colpani A, De Vito A, Barbagallo M, Madeddu G, Babudieri S, Lochoro P, Ictho J, Putoto G, Veronese N, Saracino A and Di Gennaro F. Incidence and burden of long COVID in Africa: a systematic review and meta-analysis. Sci Rep. 2023; 13:21482.
22. Beretta S, Cristillo V, Camera G, Morotti Colleoni C, Pellitteri G, Viti B, Bianchi E, Gipponi S, Grimoldi M, Valente M, Guttmann S, Cotelli M S, Palumbo P, Gelosa G, Meletti S, Schenone C, Ottaviani D, Filippi M, Zini A, Basilico P, Tancredi L, Cortelli P, Braga M, De Giuli V, Servidei S, Paolicelli D, Verde F, Caproni S, Pisani A, Lo Re V, Massacesi L, Roccatagliata D V, Manganotti P, Spitaleri D, Formenti A, Piccoli M, Marino S, Polverino P, Aguglia U, Ornello R, Perego E, Siciliano G, Merlo P, Capobianco M, Pantoni L, Lugaresi A, Angelocola S, De Rosa A, Sessa M, Beghi E, Agostoni E C, Monaco S, Padovani A, Priori A, Silani V, Tedeschi G, Ferrarese C and for Neuro C I. Incidence and Long-term Functional Outcome of Neurologic Disorders in Hospitalized Patients With COVID-19 Infected With Pre-Omicron Variants. Neurology. 2023; 101:e892-e903.
23. Tsai J, Grace A, Espinoza R and Kurian A. Incidence of long COVID and associated psychosocial characteristics in a large U.S. city. Soc Psychiatry Psychiatr Epidemiol. 2023.
24. Sedgley R, Winer-Jones J and Bonafede M. Long COVID Incidence in a Large U S Ambulatory Electronic Health Record System. Am J Epidemiol. 2023; 192:1350-1357.
25. Jacobs M M, Evans E and Ellis C. Racial, ethnic, and sex disparities in the incidence and cognitive symptomology of long COVID-19. J Natl Med Assoc. 2023; 115:233-243.
26. Gado K, Kovacs A K, Domjan G, Nagy Z Z and Bednarik G D. COVID-19 and the elderly. Physiol Int. 2022.
27. Egger M, Wimmer C, Stummer S, Reitelbach J, Bergmann J, Muller F and Jahn K. Reduced health-related quality of life, fatigue, anxiety and depression affect COVID-19 patients in the long-term after chronic critical illness. Sci Rep. 2024; 14:3016.
28. Laguarta-Val S, Varillas-Delgado D, Lizcano-Alvarez A, Molero-Sanchez A, Melian-Ortiz A, Cano-de-la-Cuerda R and Jimenez-Antona C. Effects of Aerobic Exercise Therapy through Nordic Walking Program in Lactate Concentrations, Fatigue and Quality-of-Life in Patients with Long-COVID Syndrome: A Non-Randomized Parallel Controlled Trial. J Clin Med. 2024; 13.
29. Lau B, Wentz E, Ni Z, Yenokyan K, Vergara C, Mehta S H and Duggal P. Physical Health and Mental Fatigue Disability Associated with Long COVID: Baseline Results from a US Nationwide Cohort. Am J Med. 2023.
30. Lee J S, Choi Y, Joung J Y and Son C G. Clinical and Laboratory Characteristics of Fatigue-dominant Long-COVID subjects: A Cross-Sectional Study. Am J Med. 2024.
31. Molnar T, Varnai R, Schranz D, Zavori L, Peterfi Z, Sipos D, Tokes-Fuzesi M, Illes Z, Buki A and Csecsei P. Severe Fatigue and Memory Impairment Are Associated with Lower Serum Level of Anti-SARS-CoV-2 Antibodies in Patients with Post-COVID Symptoms. J Clin Med. 2021; 10.
32. Zhang J, Shu T, Zhu R, Yang F, Zhang B and Lai X. The Long-Term Effect of COVID-19 Disease Severity on Risk of Diabetes Incidence and the Near 1-Year Follow-Up Outcomes among Postdischarge Patients in Wuhan. J Clin Med. 2022; 11.
33. Venkatesan P. NICE guideline on long COVID. Lancet Respir Med. 2021; 9:129.
34. Bello-Chavolla O Y, Fermin-Martinez C A, Ramirez-Garcia D, Vargas-Vazquez A, Fernandez-Chirino L, Basile-Alvarez M R, Sanchez-Castro P, Nunez-Luna A and Antonio-Villa N E. Prevalence and determinants of post-acute sequelae after SARS-CoV-2 infection (Long COVID) among adults in Mexico during 2022: a retrospective analysis of nationally representative data. Lancet Reg Health Am. 2024; 30:100688.
35. Chang Y Y and Wei A C. Transcriptome and machine learning analysis of the impact of COVID-19 on mitochondria and multiorgan damage. PLoS One. 2024; 19:e0297664.
36. Molnar T, Lehoczki A, Fekete M, Varnai R, Zavori L, Erdo-Bonyar S, Simon D, Berki T, Csecsei P and Ezer E. Mitochondrial dysfunction in long COVID: mechanisms, consequences, and potential therapeutic approaches. Geroscience. 2024.
37. Srinivasan K, Pandey A K, Livingston A and Venkatesh S. Roles of host mitochondria in the development of COVID-19 pathology: Could mitochondria be a potential therapeutic target? Mol Biomed. 2021; 2:38.
38. Singh K K, Chaubey G, Chen J Y and Suravajhala P. Decoding SARS-CoV-2 hijacking of host mitochondria in COVID-19 pathogenesis. Am J Physiol Cell Physiol. 2020; 319:C258-C267.
39. Ryback R and Eirin A. Mitochondria, a Missing Link in COVID-19 Heart Failure and Arrest? Front Cardiovasc Med. 2021; 8:830024.
40. Chemyak B V, Popova E N, Zinovkina L A, Lyamzaev K G and Zinovkin R A. Mitochondria as Targets for Endothelial Protection in COVID-19. Front Physiol. 2020; 11:606170.
41. Chen Z Z, Johnson L, Trahtemberg U, Baker A, Huq S, Dufresne J, Bowden P, Miao M, Ho J A, Hsu C C, Dos Santos C C and Marshall J G. Mitochondria and cytochrome components released into the plasma of severe COVID-19 and ICU acute respiratory distress syndrome patients. Clin Proteomics. 2023; 20:17.
42. Bizjak D A, Ohmayer B, Buhl J L, Schneider E M, Walther P, Calzia E, Jerg A, Matits L and Steinacker J M.

Functional and Morphological Differences of Muscle Mitochondria in Chronic Fatigue Syndrome and Post-COVID Syndrome. Int J Mol Sci. 2024; 25.
43. Bhowal C, Ghosh S, Ghatak D and De R. Pathophysiological involvement of host mitochondria in SARS-CoV-2 infection that causes COVID-19: a comprehensive evidential insight. Mol Cell Biochem. 2023; 478:1325-1343.
44. Akbari H and Taghizadeh-Hesary F. COVID-19 induced liver injury from a new perspective: Mitochondria. Mitochondrion. 2023; 70:103-110.
45. Pintos I and Soriano V. Mitochondrial damage as cause of long COVID. AIDS Rev. 2023; 26:145-149.
46. Grossini E, Concina D, Rinaldi C, Russotto S, Garhwal D, Zeppegno P, Gramaglia C, Kul S and Panella M. Association Between Plasma Redox State/Mitochondria Function and a Flu-Like Syndrome/COVID-19 in the Elderly Admitted to a Long-Tenn Care Unit. Front Physiol. 2021; 12:707587.
47. Chang X, Ismail N I, Rahman A, Xu D, Chan R W Y, Ong S G and Ong S B. Long COVID-19 and the Heart: Is Cardiac Mitochondria the Missing Link? Antioxid Redox Signal. 2023; 38:599-618.
48. Noonong K, Chatatikun M, Surinkaew S, Kotepui M, Hossain R, Bunluepuech K, Noothong C, Tedasen A, Klangbud W K, Imai M, Kawakami F, Kubo M, Kitagawa Y, Ichikawa H, Kanekura T, Sukati S, Somsak V, Udomwech L, Ichikawa T, Nissapatorn V, Tangpong J, Indo H P and Majima H J. Mitochondrial oxidative stress, mitochondrial ROS storms in long COVID pathogenesis. Front Immunol. 2023; 14:1275001.
49. Chen T H, Chang C J and Hung P H. Possible Pathogenesis and Prevention of Long COVID: SARS-CoV-2-Induced Mitochondrial Disorder. Int J Mol Sci. 2023; 24.
50. Pavli A, Theodoridou M and Maltezou H C. Post-COVID Syndrome: Incidence, Clinical Spectrum, and Challenges for Primary Healthcare Professionals. Arch Med Res. 2021; 52:575-581.
51. Ebert A, Gal E, Toth E, Szogi T, Hegyi P and Venglovecz V. Role of CFTR in diabetes-induced pancreatic ductal fluid and HCO(3) (−) secretion. J Physiol. 2024; 602:1065-1083.
52. Bodi N, Chandrakumar L, Al Doghmi A, Mezei D, Szalai Z, Barta B P, Balazs J and Bagyanszki M. Intestinal Region-Specific and Layer-Dependent Induction of TNFalpha in Rats with Streptozotocin-Induced Diabetes and after Insulin Replacement. Cells. 2021; 10.
53. Seligman M E P. Flourish: a visionary new understanding of happiness and well-being. 1st Free Press hardcover ed. New York: Free Press; 2011.
54. Metsalu T and Vilo J. ClustVis: a web tool for visualizing clustering of multivariate data using Principal Component Analysis and heatmap. Nucleic Acids Res. 2015; 43:W566-70.
55. Ishihara N, Eura Y and Mihara K. Mitofusin 1 and 2 play distinct roles in mitochondrial fusion reactions via GTPase activity. J Cell Sci. 2004; 117:6535-46.
56. Zerihun M, Sukumaran S and Qvit N. The Drp1-Mediated Mitochondrial Fission Protein Interactome as an Emerging Core Player in Mitochondrial Dynamics and Cardiovascular Disease Therapy. Int J Mol Sci. 2023; 24.
57. Friedman J R and Nunnari J. Mitochondrial form and function. Nature. 2014; 505:335-43.
58. Streng L, de Wijs C J, Raat N J H, Specht P A C, Sneiders D, van der Kaaij M, Endeman H, Mik E G and Harms F A. In Vivo and Ex Vivo Mitochondrial Function in COVID-19 Patients on the Intensive Care Unit. Biomedicines. 2022; 10.
59. Silva B S A, Pereira T, Minuzzi L G, Padilha C S, Figueiredo C, Olean-Oliveira T, Dos Santos I V M, von Ah Morano A E, Marchioto Junior O, Ribeiro J P J, Dos Santos V R, Seelaender M, Teixeira A A, Dos Santos R V T, Lemos V A, Freire A, Dorneles G P, Marmett B, Olean-Oliveira A, Teixeira M F S, Seraphim P M, Caseiro A, Pinho R A, Islam H, Little J P, Kruger K, Rosa-Neto J C, Coelho ESMJ and Lira F S. Mild to moderate post-COVID-19 alters markers of lymphocyte activation, exhaustion, and immunometabolic responses that can be partially associated by physical activity level—an observational sub-analysis fit—COVID study. Front Immunol. 2023; 14:1212745.
60. Peppercorn K, Edgar C D, Kleffmann T and Tate W P. A pilot study on the immune cell proteome of long COVID patients shows changes to physiological pathways similar to those in myalgic encephalomyelitis/chronic fatigue syndrome. Sci Rep. 2023; 13:22068.
61. Nikesjo F, Sayyab S, Karlsson L, Apostolou E, Rosen A, Hedman K and Lerm M. Defining post-acute COVID-19 syndrome (PACS) by an epigenetic biosignature in peripheral blood mononuclear cells. Clin Epigenetics. 2022; 14:172.
62. De Vitis C, Capalbo C, Torsello A, Napoli C, Salvati V, Loffredo C, Blandino G, Piaggio G, Auciello F R, Pelliccia F, Salerno G, Simmaco M, Di Magno L, Canettieri G, Coluzzi F, Mancini R, Rocco M and Sciacchitano S. Opposite Effect of Thyroid Hormones on Oxidative Stress and on Mitochondrial Respiration in COVID-19 Patients. Antioxidants (Basel). 2022; 11.
63. Ernst T, Ryan M C, Liang H J, Wang J P, Cunningham E, Saleh M G, Kottilil S and Chang L. Neuronal and Glial Metabolite Abnormalities in Participants With Persistent Neuropsychiatric Symptoms After COVID-19: A Brain Proton Magnetic Resonance Spectroscopy Study. J Infect Dis. 2023; 228:1559-1570.
64. Ranisavljev M, Todorovic N, Ostojic J and Ostojic S M. Reduced tissue creatine levels in patients with long COVID-19: A cross-sectional study. J Postgrad Med. 2023; 69:162-163.
65. Holmes E, Wist J, Masuda R, Lodge S, Nitschke P, Kimhofer T, Loo R L, Begum S, Boughton B, Yang R, Morillon A C, Chin S T, Hall D, Ryan M, Bong S H, Gay M, Edgar D W, Lindon J C, Richards T, Yeap B B, Pettersson S, Spraul M, Schaefer H, Lawler N G, Gray N, Whiley L and Nicholson J K. Incomplete Systemic Recovery and Metabolic Phenoreversion in Post-Acute-Phase Nonhospitalized COVID-19 Patients: Implications for Assessment of Post-Acute COVID-19 Syndrome. J Proteome Res. 2021; 20:3315-3329.
66. Finnigan L E M, Cassar M P, Koziel M J, Pradines J, Lamlum H, Azer K, Kirby D, Montgomery H, Neubauer S, Valkovic L and Raman B. Efficacy and tolerability of an endogenous metabolic modulator (AXA1125) in fatigue-predominant long COVID: a single-centre, double-blind, randomised controlled phase 2a pilot study. EClinicalMedicine. 2023; 59:101946.
67. Jamieson A, Al Saikhan L, Alghamdi L, Hamill Howes L, Purcell H, Hillman T, Heightman M, Treibel T, Orini M, Bell R, Scully M, Hamer M, Chaturvedi N, Montgomery H, Hughes A D, Astin R and Jones S. Mechanisms underlying exercise intolerance in long COVID: An accumulation of multisystem dysfunction. Physiol Rep. 2024; 12:e15940.
68. Karim A, Muhammad T, Iqbal M S and Qaisar R. Elevated plasma CAF22 are incompletely restored six months after COVID-19 infection in older men. Exp Gerontol. 2023; 171:112034.
69. Mikuteit M, Baskal S, Klawitter S, Dopfer-Jablonka A, Behrens G M N, Muller F, Schroder D, Klawonn F, Steffens S and Tsikas D. Amino acids, post-translational modifications, nitric oxide, and oxidative stress in serum and urine of long COVID and ex COVID human subjects. Amino Acids. 2023; 55:1173-1188.
70. Vollbracht C and Kraft K. Oxidative Stress and Hyper-Inflammation as Major Drivers of Severe COVID-19 and Long COVID: Implications for the Benefit of High-Dose Intravenous Vitamin C. Front Pharmacol. 2022; 13:899198.
71. Trimarco V, Izzo R, Mone P, Trimarco B and Santulli G. Targeting endothelial dysfunction and oxidative stress in Long-COVID. Pharmacol Res. 2022; 184:106451.
72. Mrakic-Sposta S, Vezzoli A, Garetto G, Paganini M, Camporesi E, Giacon T A, Dellanoce C, Agrimi J and Bosco G. Hyperbaric Oxygen Therapy Counters Oxidative Stress/Inflammation-Driven Symptoms in Long COVID-19 Patients: Preliminary Outcomes. Metabolites. 2023; 13.
73. Juhász P, Bohus P, Sipos A, Curtin N, Mehes G and Bai P. Oxidative stress and PARP activation in the lungs is an early event in COVID-19 pneumonia. medRxiv. 2024:2024.09.03.24312996.
74. Mehrzadi S, Karimi M Y, Fatemi A, Reiter R J and Hosseinzadeh A. SARS-CoV-2 and other coronaviruses negatively influence mitochondrial quality control: beneficial effects of melatonin. Pharmacol Ther. 2021; 224:107825.
75. Khan M, Syed G H, Kim S J and Siddiqui A. Mitochondrial dynamics and viral infections: A close nexus. Biochim Biophys Acta. 2015; 1853:2822-33.
76. Valdes-Aguayo J J, Garza-Veloz I, Vargas-Rodriguez J R, Martinez-Vazquez M C, Avila-Carrasco L, Bernal-Silva S, Gonzalez-Fuentes C, Comas-Garcia A, Alvarado-Hernandez D E, Centeno-Ramirez A S H, Rodriguez-Sanchez I P, Delgado-Enciso I and Martinez-Fierro M L. Peripheral Blood Mitochondrial DNA Levels Were Modulated by SARS-CoV-2 Infection Severity and Its Lessening Was Associated With Mortality Among Hospitalized Patients With COVID-19. Front Cell Infect Microbiol. 2021; 11:754708.
77. Scozzi D, Cano M, Ma L, Zhou D, Zhu J H, O'Halloran J A, Goss C, Rauseo A M, Liu Z, Sahu S K, Peritore V, Rocco M, Ricci A, Amodeo R, Aimati L, Ibrahim M, Hachem R, Kreisel D, Mudd P A, Kulkarni H S and Gelman A E. Circulating mitochondrial DNA is an early indicator of severe illness and mortality from COVID-19. JCI Insight. 2021; 6.
78. Valdes-Aguayo J J, Garza-Veloz I, Badillo-Almaraz J I, Bernal-Silva S, Martinez-Vazquez M C, Juarez-Alcala V, Vargas-Rodriguez J R, Gaeta-Velasco M L, Gonzalez-Fuentes C, Avila-Carrasco L and Martinez-Fierro M L. Mitochondria and Mitochondrial DNA: Key Elements in the Pathogenesis and Exacerbation of the Inflammatory State Caused by COVID-19. Medicina (Kaunas). 2021; 57.
79. Archer S L, Dasgupta A, Chen K H, Wu D, Baid K, Mamatis J E, Gonzalez V, Read A, Bentley R E, Martin A Y, Mewburn J D, Dunham-Snary K J, Evans G A, Levy G, Jones O, Al-Qazazi R, Ring B, Alizadeh E, Hindmarch C C, Rossi J, Lima P D, Falzarano D, Banerjee A and Colpitts C C. SARS-CoV-2 mitochondriopathy in COVID-19 pneumonia exacerbates hypoxemia. Redox Biol. 2022; 58:102508.
80. Nailwal H and Chan F K. Necroptosis in anti-viral inflammation. Cell Death Differ. 2019; 26:4-13.
81. Zhang Q, Raoof M, Chen Y, Sumi Y, Sursal T, Junger W, Brohi K, Itagaki K and Hauser C J. Circulating mitochondrial DAMPs cause inflammatory responses to injury. Nature. 2010; 464:104-7.
82. Tilokani L, Nagashima S, Paupe V and Prudent J. Mitochondrial dynamics: overview of molecular mechanisms. Essays Biochem. 2018; 62:341-360.
83. Siekacz K, Kumor-Kisielewska A, Milkowska-Dymanowska J, Pietrusinska M, Bartczak K, Majewski S, Stanczyk A, Piotrowski W J and Bialas A J. Oxidative Biomarkers Associated with the Pulmonary Manifestation of Post-COVID-19 Complications. J Clin Med. 2023; 12.
84. Versace V and Tankisi H. Long-COVID and myalgic encephalomyelitis/chronic fatigue syndrome (ME/CFS): Potential neurophysiological biomarkers for these enigmatic entities. Clin Neurophysiol. 2023; 147:58-59.
85. Tziastoudi M, Cholevas C, Stefanidis I and Theoharides T C. Genetics of COVID-19 and myalgic encephalomyelitis/chronic fatigue syndrome: a systematic review. Ann Clin Transl Neurol. 2022; 9:1838-1857.
86. McLaughlin M, Sanal-Hayes N E M, Hayes L D, Berry E C and Sculthorpe N F. People with Long COVID and Myalgic Encephalomyelitis/Chronic Fatigue Syndrome Exhibit Similarly Impaired Vascular Function. Am J Med. 2023.
87. Bonilla H, Quach T C, Tiwari A, Bonilla A E, Miglis M, Yang P C, Eggert L E, Sharifi H, Horomanski A, Subramanian A, Smirnoff L, Simpson N, Halawi H, Sum-Ping O, Kalinowski A, Patel Z M, Shafer R W and Geng L N. Myalgic Encephalomyelitis/Chronic Fatigue Syndrome is common in post-acute sequelae of SARS-CoV-2 infection (PASC): Results from a post-COVID-19 multidisciplinary clinic. Front Neurol. 2023; 14:1090747.
88. Sotzny F, Filgueiras I S, Kedor C, Freitag H, Wittke K, Bauer S, Sepulveda N, Mathias da Fonseca D L, Baiocchi G C, Marques A H C, Kim M, Lange T, Placa D R, Luebber F, Paulus F M, De Vito R, Jurisica I, Schulze-Forster K, Paul F, Bellmann-Strobl J, Rust R, Hoppmann U, Shoenfeld Y, Riemekasten G, Heidecke H, Cabral-Marques O and Scheibenbogen C. Dysregulated autoantibodies targeting vaso- and immunoregulatory receptors in Post COVID Syndrome correlate with symptom severity. Front Immunol. 2022; 13:981532.
89. Fagyas M, Nagy B, Jr., Raduly A P, Manyine I S, Martha L, Erdosi G, Sipka S, Jr., Enyedi E, Szabo A A, Polik Z, Kappelmayer J, Papp Z, Borbely A, Szabo T, Balla J, Balla G, Bai P, Bacsi A and Toth A. The majority of severe COVID-19 patients develop anti-cardiac autoantibodies. Geroscience. 2022:1-14.
90. Seibert F S, Stervbo U, Wiemers L, Skrzypczyk S, Hogeweg M, Bertram S, Kurek J, Anft M, Westhoff T H and Babel N. Severity of neurological Long-COVID symptoms correlates with increased level of autoantibodies targeting vasoregulatory and autonomic nervous system receptors. Autoimmun Rev. 2023; 22:103445.
91. Nersesjan V, Amiri M, Nilsson A C, Wamberg C, Jensen V V S, Petersen C B, Hejl A M, Lebech A M, Theut A M, Jorgensen C S, Blaabjerg M, Benros M E and Kondziella D. SARS-CoV-2 and autoantibodies in the cerebrospinal fluid of COVID-19 patients: prospective multicentre cohort study. Brain Commun. 2023; 5:fcad274.
92. Lee S J, Yoon T, Ha J W, Kim J, Lee K H, Lee J A, Kim C H, Lee S W, Kim J H, Ahn J Y, Ku N S, Choi J Y, Yeom J S and Jeong S J. Prevalence, clinical significance, and persistence of autoantibodies in COVID-19. Virol J. 2023; 20:236.
93. Fonseca D L M, Filgueiras I S, Marques A H C, Vojdani E, Halpert G, Ostrinski Y, Baiocchi G C, Placa D R, Freire P P, Pour S Z, Moll G, Catar R, Lavi Y B, Silverberg J I, Zimmerman J, Cabral-Miranda G, Carvalho R F, Khan T A, Heidecke H, Dalmolin R J S, Luchessi A D, Ochs H D, Schimke L F, Amital H, Riemekasten G, Zyskind I, Rosenberg A Z, Vojdani A, Shoenfeld Y and Cabral-Marques O. Severe COVID-19 patients exhibit elevated levels of autoantibodies targeting cardiolipin and platelet glycoprotein with age: a systems biology approach. NPJ Aging. 2023; 9:21.
94. Credle J J, Gunn J, Sangkhapreecha P, Monaco D R, Zheng X A, Tsai H J, Wilbon A, Morgenlander W R, Rastegar A, Dong Y, Jayaraman S, Tosi L, Parekkadan B, Baer A N, Roederer M, Bloch E M, Tobian A A R, Zyskind I, Silverberg J I, Rosenberg A Z, Cox A L, Lloyd T, Mammen A L and Benjamin Larman H. Unbiased discovery of autoantibodies associated with severe COVID-19 via genome-scale self-assembled DNA-barcoded protein libraries. Nat Biomed Eng. 2022; 6:992-1003.
95. Casciola-Rosen L, Thiemann D R, Andrade F, Trejo-Zambrano M I, Leonard E K, Spangler J B, Skinner N E, Bailey J, Yegnasubramanian S, Wang R, Vaghasia A M, Gupta A, Cox A L, Ray S C, Linville R M, Guo Z, Searson P C, Machamer C E, Desiderio S, Sauer L M, Laeyendecker O, Garibaldi B T, Gao L, Damarla M, Hassoun P M, Hooper J E, Mecoli C A, Christopher-Stine L, Gutierrez-Alamillo L, Yang Q, Hines D, Clarke W A, Rothman R E, Pekosz A, Fenstermacher K Z, Wang Z, Zeger S L and Rosen A. IgM anti-ACE2 autoantibodies in severe COVID-19 activate complement and perturb vascular endothelial function. JCI Insight. 2022; 7.
96. Cabral-Marques O, Halpert G, Schimke L F, Ostrinski Y, Vojdani A, Baiocchi G C, Freire P P, Filgueiras I S, Zyskind I, Lattin M T, Tran F, Schreiber S, Marques A H C, Placa D R, Fonseca D L M, Humrich J Y, Muller A, Giil L M, Grasshoff H, Schumann A, Hackel A, Junker J, Meyer C, Ochs H D, Lavi Y B, Scheibenbogen C, Dechend R, Jurisica I, Schulze-Forster K, Silverberg J I, Amital H, Zimmerman J, Heidecke H, Rosenberg A Z, Riemekasten G and Shoenfeld Y. Autoantibodies targeting GPCRs and RAS-related molecules associate with COVID-19 severity. Nat Commun. 2022; 13:1220.
97. Di Florio D N, Beetler D J, McCabe E J, Sin J, Ikezu T and Fairweather D. Mitochondrial extracellular vesicles, autoimmunity and myocarditis. Front Immunol. 2024; 15:1374796.
98. Montenegro Y H A, Bobermin L D, Sesterheim P, Salvato R S, Anschau F, de Oliveira M J S, Wyse A T S, Netto C A, Goncalves C S, Quincozes-Santos A and Leipnitz G. Serum of COVID-19 patients changes neuroinflammation and mitochondrial homeostasis markers in hippocampus of aged rats. J Neurovirol. 2023; 29:577-587.
99. Vojdani A, Almulla A F, Zhou B, Al-Hakeim H K and Maes M. Reactivation of herpesvirus type 6 and IgA/IgM-mediated responses to activin-A underpin long COVID, including affective symptoms and chronic fatigue syndrome. Acta Neuropsychiatr. 2024; 36:172-184.
100. Tassaneeyasin T, Sungkanuparph S, Srichatrapimuk S, Charoensri A, Thammavaranucupt K, Jayanama K and Kirdlarp S. Prevalence and risk factors of cytomegalovirus reactivation in critically Ill patients with COVID-19 pneumonia. PLoS One. 2024; 19:e0303995.
101. Talukder S, Deb P, Parveen M, Zannat K E, Bhuiyan A H, Yeasmin M, Molla M M A and Saif-Ur-Rahman K M. Clinical features and outcomes of COVID-19 patients with concomitant herpesvirus co-infection or reactivation: A systematic review. New Microbes New Infect. 2024; 58:101233.
102. Payen S H, Adhikari K, Petereit J, Uppal T, Rossetto C C and Verma S C. SARS-CoV-2 superinfection in CD14(+) monocytes with latent human cytomegalovirus (HCMV) promotes inflammatory cascade. Virus Res. 2024; 345:199375.
103. Mattei A, Schiavoni L, Riva E, Ciccozzi M, Veralli R, Urselli A, Citriniti V, Nenna A, Pascarella G, Costa F, Cataldo R, Agro F E and Carassiti M. Epstein-Barr virus, Cytomegalovirus, and Herpes Simplex-1/2 reactivations in critically ill patients with COVID-19. Intensive Care Med Exp. 2024; 12:40.
104. Grubelnik G, Korva M, Kogoj R, Polanc T, Mavric M, Jevsnik Virant M, Ursic T, Kese D, Seme K, Petrovec M, Jereb M and Avsic-Zupanc T. Herpesviridae and Atypical Bacteria Co-Detections in Lower Respiratory Tract Samples of SARS-CoV-2-Positive Patients Admitted to an Intensive Care Unit. Microorganisms. 2024; 12.
105. Haddad M, Sheybani F, Olfati N, Nahayati M A, Boostani R, Layegh P and Rashid-Nejad A. Central nervous system reactivation of herpesviridae family in patients with COVID-19. J Neurovirol. 2023; 29:211-217.
106. Giacconi R, Cardelli M, Piacenza F, Pierpaoli E, Farnocchia E, Di Rosa M, Bonfigli A R, Casoli T, Marchegiani F, Marcheselli F, Recchioni R, Stripoli P, Galeazzi R, Cherubim A, Fedecostante M, Sarzani R, Di Pentima C, Giordano P, Antonicelli R, Provinciali M and Lattanzio F. Effect of Cytomegalovirus Reactivation on Inflammatory Status and Mortality of Older COVID-19 Patients. Int J Mol Sci. 2023; 24.
107. Bernal K D E and Whitehurst C B. Incidence of Epstein-Barr virus reactivation is elevated in COVID-19 patients. Virus Res. 2023; 334:199157.
108. Banko A, Miljanovic D and Cirkovic A. Systematic review with meta-analysis of active herpesvirus infections in patients with COVID-19: Old players on the new field. Int J Infect Dis. 2023; 130:108-125.
109. Reizine F, Liard C, Pronier C, Thibault V, Maamar A, Gacouin A and Tadie J M. Herpesviridae systemic reactivation in patients with COVID-19-associated ARDS. J Hosp Infect. 2022; 119:189-191.
110. Lino K, Alves L S, Raposo J V, Medeiros T, Souza C F, Silva A A D, de Paula V S and Almeida J R. Presence and clinical impact of human herpesvirus-6 infection inpatients with moderate to critical coronavirus disease-19. J Med Virol. 2022; 94:1212-1216.
111. Chen J, Song J, Dai L, Post S R and Qin Z. SARS-CoV-2 infection and lytic reactivation of herpesviruses: A potential threat in the postpandemic era? J Med Virol. 2022; 94:5103-5111.
112. Brooks B, Tancredi C, Song Y, Mogus A T, Huang M W, Zhu H, Phan T L, Zhu H, Kadl A, Woodfolk J, Jerome K R and Zeichner S L. Epstein-Barr Virus and Human Herpesvirus-6 Reactivation in Acute COVID-19 Patients. Viruses. 2022; 14.
113. Chen T, Song J, Liu H, Zheng H and Chen C. Positive Epstein-Barr virus detection in coronavirus disease 2019 (COVID-19) patients. Sci Rep. 2021; 11:10902.
114. Frozza F T B, Fazolo T, de Souza P O, Lima K, da Fontoura J C, Borba T S, Polese-Bonatto M, Kern L B, Stein R T, Pawelec G and Bonorino C. A high CMV-specific T cell response associates with SARS-CoV-2-specific IL-17 T cell production. Med Microbiol Immunol. 2023; 212:75-91.
115. Liu Z, Hollmann C, Kalanidhi S, Grothey A, Keating S, Mena-Palomo I, Lamer S, Schlosser A, Kaiping A, Scheller C, Sotzny F, Horn A, Nurnberger C, Cejka V, Afshar B, Bahmer T, Schreiber S, Vehreschild J J, Miljukov O, Schafer C, Kretzler L, Keil T, Reese J P, Eichner F A, Schmidbauer L, Heuschmann P U, Stork S, Morbach C, Riemekasten G, Beyersdorf N, Scheibenbogen C, Naviaux R K, Williams M, Ariza M E and Prusty B K. Increased circulating fibronectin, depletion of natural IgM and heightened EBV, HSV-1 reactivation in ME/CFS and long COVID. medRxiv. 2023.
116. Kiss T, Tarantini S, Csipo T, Balasubramanian P, Nyul-Toth A, Yabluchanskiy A, Wren J D, Garman L, Huffman D M, Csiszar A and Ungvari Z. Circulating anti-geronic factors from heterochonic parabionts promote vascular rejuvenation in aged mice: transcriptional footprint of mitochondrial protection, attenuation of oxidative stress, and rescue of endothelial function by young blood. Geroscience. 2020; 42:727-748.
117. Kiss T, Nyul-Toth A, Balasubramanian P, Tarantini S, Ahire C, Yabluchanskiy A, Csipo T, Farkas E, Wren J D, Garman L, Csiszar A and Ungvari Z. Nicotinamide mononucleotide (NMN) supplementation promotes neurovascular rejuvenation in aged mice: transcriptional footprint of SIRT1 activation, mitochondrial protection, anti-inflammatory, and anti-apoptotic effects. Geroscience. 2020.
118. Tarantini S, Valcarcel-Ares M N, Toth P, Yabluchanskiy A, Tucsek Z, Kiss T, Hertelendy P, Kinter M, Ballabh P, Sule Z, Farkas E, Baur J A, Sinclair D A, Csiszar A and Ungvari Z. Nicotinamide mononucleotide (NMN) supplementation rescues cerebromicrovascular endothelial function and neurovascular coupling responses and improves cognitive function in aged mice. Redox Biol. 2019; 24:101192.
119. Csiszar A, Yabluchanskiy A, Ungvari A, Ungvari Z and Tarantini S. Overexpression of catalase targeted to mitochondria improves neurovascular coupling responses in aged mice. Geroscience. 2019; 41:609-617.
120. Tarantini S, Valcarcel-Ares N M, Yabluchanskiy A, Fulop G A, Hertelendy P, Gautam T, Farkas E, Perz A, Rabinovitch P S, Sonntag W E, Csiszar A and Ungvari Z. Treatment with the mitochondrial-targeted antioxidant peptide SS-31 rescues neurovascular coupling responses and cerebrovascular endothelial function and improves cognition in aged mice. Aging Cell. 2018; 17.
121. Saleh J, Peyssonnaux C, Singh K K and Edeas M. Mitochondria and microbiota dysfunction in COVID-19 pathogenesis. Mitochondrion. 2020; 54:1-7.
122. Georgieva E, Ananiev J, Yovchev Y, Arabadzhiev G, Abrashev H, Abrasheva D, Atanasov V, Kostandieva R, Mitev M, Petkova-Parlapanska K, Karamalakova Y, Koleva-Korkelia I, Tsoneva V and Nikolova G. COVID-19 Complications: Oxidative Stress, Inflammation, and Mitochondrial and Endothelial Dysfunction. Int J Mol Sci. 2023; 24.
123. Lin M M, Liu N, Qin Z H and Wang Y. Mitochondrial-derived damage-associated molecular patterns amplify neuroinflammation in neurodegenerative diseases. Acta Pharmacol Sin. 2022; 43:2439-2447.
124. Mantle D, Hargreaves I P, Domingo J C and Castro-Marrero J. Mitochondrial Dysfunction and Coenzyme Q10 Supplementation in Post-Viral Fatigue Syndrome: An Overview. Int J Mol Sci. 2024; 25.
125. Kow C S, Ramachandram D S and Hasan S S. Coenzyme Q10 therapy in patients with post COVID-19 condition. Lancet Reg Health Eur. 2023; 25:100567.
126. Hansen K S, Mogensen T H, Agergaard J, Schiottz-Christensen B, Ostergaard L, Vibholm L K and Leth S. High-dose coenzyme Q10 therapy versus placebo in patients with post COVID-19 condition: a randomized, phase 2, crossover trial. Lancet Reg Health Eur. 2023; 24:100539.

1. A method for in vitro diagnosis of Long COVID in a subject, wherein said method comprises the following steps:
a) providing a biological sample obtained from the subject,
b) measuring the levels of at least one protein in said sample,
wherein the at least one protein is selected from Autophagy Related 4B Cysteine Peptidase (ATG4B), Mitofusin 2 (MFN2), Dynamin-related Protein 1 (DRP1), and/or Superoxide dismutase 1 (SOD1),
c) comparing the levels of the at least one protein measured in step b) with a respective reference, wherein an increase in the levels of the at least one protein in said sample relative to the reference is indicative of Long COVID diagnosis.

2. The method according to claim 1, wherein the at least one protein is at least two proteins selected from ATG4B, MFN2, DRP1, and/or SOD1, and wherein an increase in the levels of at least one of the proteins in said sample relative to the respective reference is indicative of Long COVID diagnosis.

3. The method according to claim 1, wherein the at least one protein comprises the following four proteins: ATG4B, MFN2, DRP1, and/or SOD1; and wherein an increase in the levels of at least one of the proteins in said sample relative to the respective reference is indicative of Long COVID diagnosis.

4. A method of monitoring the treatment of Long COVID in a subject, wherein said method comprises the following steps:
a) providing at least one biological sample obtained from the subject undergoing Long COVID treatment,
wherein the sample(s) are obtained after the long COVID treatment has begun,
b) measuring the levels of at least one protein in said sample(s),
wherein the at least one protein is selected from ATG4B, MFN2, DRP1, and/or SOD1,
c) comparing the levels of the at least one protein measured in step b) with a reference, wherein a similarity in the levels of the at least one protein in said sample relative to the reference indicates an effective Long COVID treatment.

5. The method according to claim 4, wherein the at least one protein is at least two proteins selected from ATG4B, MFN2, DRP1, and/or SOD1.

6. A method of in vitro monitoring the treatment of Long COVID in a subject, wherein said method comprises the following steps:
a) providing at least two biological samples obtained from the subject undergoing Long COVID treatment,
wherein one sample is obtained prior to the start of treatment and the other sample(s) are obtained after the treatment has begun,
b) measuring the levels of at least one protein in the sample obtained prior to the start of the treatment,
wherein the at least one protein is selected from ATG4B, MFN2, DRP1, and/or SOD1,
thereby obtaining a baseline,
c) measuring the levels of the same protein(s) as in step b) in the sample(s) obtained after the treatment has begun,
d) comparing the levels of the protein(s) measured in step c) with the baseline obtained in step b), wherein a decrease in the levels of the protein(s) in the sample(s) obtained after the treatment has begun, relative to the baseline, indicates an effective Long COVID treatment.

7. The method according to claim 6, wherein the at least one protein is at least two proteins selected from ATG4B, MFN2, DRP1, and/or SOD1

8. The method according to claim 1, wherein the biological sample is a tissue sample.

9. The method according to claim 8, wherein said biological sample is a biopsy sample.

10. The method according to claim 1, wherein said measuring of the levels of at least one protein selected from ATG4B, MFN2, DRP1, and/or SOD1 in said sample is performed with a kit comprising at least one means for measuring the levels of said at least one protein.

11. The method according to claim 10, wherein said means for measuring the levels of at least one protein comprises antibodies against said at least one protein.

12. A method for treating long COVID in a subject comprising facilitating mitochondrial regeneration in said subject by administrating an agent that directly or indirectly decreases the levels of at least one protein selected from ATG4B, MFN2, DRP1, and/or SOD1.

13. The method according to claim 12, wherein said agent is selected from coenzyme Q10 (CoQ10 or Q10), rapamycin, metformin, mdivi-1 (mitochondrial division inhibitor), P110 (a Drp1-derived peptide), thiazolidinediones, pioglitazone, thiamine, idebenone, imeglimin, bezafibrate, epicatechin, alpha-lipoic acid, resveratrol, riboflavin, dichloroacetate, DRP1 modulators, MFN2 modulators, ATG4B modulators, Q1067, MitoQ (NCT05373043), and/or nicotinamide riboside (NCT05703074).

14. The method according to claim 13, wherein said agent is selected from coenzyme Q10 (CoQ10 or Q10), rapamycin, and/or metformin.


SEQUENCE LISTING (10 sequences)

SEQ ID NO: 1 - DNA, 20 bp, synthetic construct
  source 1..20: mol_type = other DNA; note = MTCYTB forward primer
agccaaccccttaaacaccc

SEQ ID NO: 2 - DNA, 20 bp, synthetic construct
  source 1..20: mol_type = other DNA; note = MTCYTB reverse primer
tcattcgggcttgatgtggg

SEQ ID NO: 3 - DNA, 20 bp, synthetic construct
  source 1..20: mol_type = other DNA; note = MTND4 forward primer
cctgactcctacccctcaca

SEQ ID NO: 4 - DNA, 20 bp, synthetic construct
  source 1..20: mol_type = other DNA; note = MTND4 reverse primer
tggataagtggcgttggctt

SEQ ID NO: 5 - DNA, 21 bp, synthetic construct
  source 1..21: mol_type = other DNA; note = MTATP6 forward primer
cccacttcttaccacaaggca

SEQ ID NO: 6 - DNA, 20 bp, synthetic construct
  source 1..20: mol_type = other DNA; note = MTATP6 reverse primer
tggggataaggggtgtaggt

SEQ ID NO: 7 - DNA, 20 bp, synthetic construct
  source 1..20: mol_type = other DNA; note = MTND5 forward primer
accacatcatcgaaaccgca

SEQ ID NO: 8 - DNA, 20 bp, synthetic construct
  source 1..20: mol_type = other DNA; note = MTND5 reverse primer
gatagggctcaggcgtttgt

SEQ ID NO: 9 - DNA, 20 bp, synthetic construct
  source 1..20: mol_type = other DNA; note = MTND1 forward primer
aaagagcccctaaaacccgc

SEQ ID NO: 10 - DNA, 21 bp, synthetic construct
  source 1..21: mol_type = other DNA; note = MTND1 reverse primer
cggtgatgtagagggtgatgg